(19) United States
(12) Patent Application Publication
(10) Pub. No.: US 2026/0165852 A1
(43) Pub. Date: Jun. 18, 2026

(54) PARALLEL JAW INSERTER

(71) Applicant: Warsaw Orthopedic, Inc., Warsaw, IN (US)

(72) Inventors: Darren Davis, Memphis, TN (US); Cristian Capote, Memphis, TN (US); Patrick Keene, Memphis, TN (US); John Hall, Germantown, TN (US); Robert Loke, Memphis, TN (US); Stanley Kyle Hayes, Mission Viejo, CA (US)

(73) Assignee: Warsaw Orthopedic, Inc., Warsaw, IN (US)

(21) Appl. No.: 19/386,772

(22) Filed: Nov. 12, 2025

Related U.S. Application Data
(63) Continuation of application No. 18/322,353, filed on May 23, 2023, now Pat. No. 12,485,019.
Application No. 18/322,353 is a continuation-in-part of application No. 17/736,523, filed on May 4, 2022, now Pat. No. 12,414,863.
Application No. 17/736,523 is a continuation-in-part of application No. 17/665,449, filed on Feb. 4, 2022, now Pat. No. 12,440,349.
Application No. 17/665,449 is a continuation-in-part of application No. 17/515,709, filed on Nov. 1, 2021, now Pat. No. 12,295,865.
Application No. 17/515,709 is a continuation-in-part of application No. 17/356,950, filed on Jun. 24, 2021, now Pat. No. 11,612,499.

(51) Int. Cl. A61F 2/46; A61F 2/30; A61F 2/44

(52) U.S. Cl. CPC A61F 2/4611; A61F 2/4455; A61F 2002/30387; A61F 2002/30523; A61F 2002/3054; A61F 2002/30579; A61F 2002/4628

(57) ABSTRACT

An interbody system including an implant and a tool for inserting and expanding the medical implant and locking the implant in place is disclosed. The medical implant may include an expandable body defined by a superior endplate and an inferior endplate that are hingedly coupled and may be expanded and lordosed. The superior and inferior endplate may include gripping protrusions that mate and/or directly engage with recessed jaws of disclosed surgical tool. The surgical tool may include a vertically movable jaw to expand and contract the implant when engaged with the gripping protrusions of the implant. The surgical tool may also include a drive portion that engages the drive feature of the lock screw with the drive end of the surgical tool lock the implant while in an expanded position.

### CROSS-REFERENCE TO RELATED APPLICATIONS

This application is a continuation of: U.S. patent application Ser. No. 18/322,353, titled PARALLEL JAW INSERTER, filed May 23, 2023, which is a continuation in part of: U.S. patent application Ser. No. 17/736,523, titled EXPANDABLE IMPLANT AND CORRESPONDING SURGICAL TOOL, filed May 4, 2022 (now U.S. Pat. No. 12,414,863), which is a continuation in part of U.S. patent application Ser. No. 17/665,449, titled EXPANDABLE IMPLANT AND CORRESPONDING INSERTER, filed Feb. 4, 2022 (now U.S. Pat. No. 12,440,349), which is a continuation in part of U.S. patent application Ser. No. 17/515,709, titled EXPANDABLE IMPLANT AND CORRESPONDING INSERTER, filed Nov. 1, 2021 (now U.S. Pat. No. 12,295,865), which is a continuation in part of U.S. patent application Ser. No. 17/356,950, titled EXPANDABLE INTERBODY IMPLANT, filed Jun. 24, 2021 (now U.S. Pat. No. 11,612,499). The entire disclosure of each application is incorporated herein by reference. This application also incorporates by reference U.S. application Ser. No. 17/307,578, titled EXTERNALLY DRIVEN EXPANDABLE INTERBODY AND RELATED METHODS, filed May 5, 2021; U.S. Pat. No. 11,096,796, titled INTERBODY SPINAL IMPLANT HAVING A ROUGHENED SURFACE TOPOGRAPHY ON ONE OR MORE INTERNAL SURFACES, and filed on March, 4, 2013; and U.S. Pat. No. 10,821,000, titled TITANIUM IMPLANT SURFACES FREE FROM ALPHA CASE AND WITH ENHANCED OSTEOINDUCTION, and filed Jun. 29, 2017.

### FIELD

The present technology is generally related to an externally driven expandable interbody implant for use in a medical procedure related to the spine. In some embodiments, disclosed implants may be used in an anterior cervical discectomy and fusion (ACDF) procedure although other uses in other areas of the spine or between two bones are also contemplated.

### BACKGROUND

Mechanically operated interbody implants may be used to align and/or realign a patient's spine during a medical procedure and/or for purposes of fusion, degenerative tissue and/or trauma/repair procedures. Conventional implants designed for the Thoracic and Lumbar region of the spine often include top and bottom endplates and a mechanical means to separate the top and bottom endplates. The mechanical mechanisms to separate the top and bottom endplates are often cumbersome and require a large footprint that is often unsuitable, for example, for ACDF type surgeries of the cervical portion of the spine.

Many currently available ACDF type implants may be limited in an ability to optimize the adjustment of lordosis or sagittal alignment of the vertebral bodies because they may rely on a fixed lordotic angle between the superior/cephalad and inferior/caudad faces of the device.

### SUMMARY

The techniques of this disclosure generally relate to an expandable interbody implant including a superior endplate and an inferior endplate hingedly coupled and which may further include a locking element to secure the inferior endplate and superior endplate in a particular configuration, for example. The superior and inferior endplates may be moved in a multitude of expanded and/or lordosed or kyphosed or otherwise angled configurations via an external inserter, for example. In various embodiments, a locking screw may include a drive feature for engaging with a drive end of a surgical tool. In various embodiments, gripping protrusions on the proximal end of the implant may be used for gripping the implant to insert it into a disc space and afterward the implant may be expanded by a corresponding drive tool. Additionally, in various embodiments, the locking screw may also be used to grip and stabilize the implant to insert the implant into a disc space. Additionally, in various embodiments female recesses, rather than tangs (or male bosses or protrusions), may be used for gripping the implant and inserting the implant into a disc space.

In one aspect, the present disclosure provides for an expandable implant movable between a contracted position (closed position) and an expanded position, for example. The expandable implant may include an expandable body extending from a proximal end to a distal end in a proximal-to-distal direction (may also be referred to as an anterior-to-posterior direction depending on surgical technique), extending from a first lateral side to a second lateral side in a widthwise direction, and extending from a superior endplate to an inferior endplate in a heightwise direction (may also be referred to as a cephalad-to-caudal and/or vertical direction depending on surgical technique), for example. In various embodiments, the expandable body may be defined by a superior endplate and an inferior endplate that are hingedly connected, for example. In various embodiments, the superior endplate includes a first core having a distal engagement surface (may also be referred to as a posterior engagement surface depending on surgical technique) and the inferior endplate includes a second core having a proximal engagement surface (may also be referred to as an anterior engagement surface) and a threaded screw aperture, for example. In various embodiments, disclosed implants may include a locking screw, having a drive feature that may engage with the drive end of a surgical tool and be movable between a locked position and an unlocked position, for example. In various embodiments, when in the locked position, the locking screw urges the distal engagement surface of the first core into direct contact with the proximal engagement surface of the second core, for example. In this way, a surgeon may expand the implant to an appropriate height and angle, and then the superior endplate and inferior endplate may be locked into an angled configuration relative to one another.

In another aspect, the disclosure provides for a system including a medical implant and a surgical tool, for example. The system may include an expandable implant movable between a contracted position and an expanded position, for example. In various embodiments, the expanded position may also refer to a distracted and angled orientation of the superior endplate and inferior endplate. The expandable implant may include an expandable body extending from a proximal end to a distal end in a proximal-to-distal direction and extending from a first lateral side to a second lateral side in a widthwise direction, for example. In various embodiments, the expandable body may be defined by a superior endplate and an inferior endplate that are hingedly connected, for example. In various embodiments, the superior endplate includes a first core having a distal engagement surface and the inferior endplate includes a second core having a proximal engagement surface and a threaded screw aperture, for example. In various embodiments, disclosed implants may include a locking screw that is disposed in the threaded screw aperture and movable between a locked position and an unlocked position, for example. In various embodiments, when in the locked position, the locking screw urges the distal engagement surface of the first core into direct contact with the proximal engagement surface of the second core, for example. The system may also include a surgical tool for expanding the implant and tightening the locking screw while the implant is expanded at a desired height, position, and/or angle. In various embodiments, the surgical tool may include an inserter portion having a rotating and a non-rotating jaws that engages with the gripping protrusions of the disclosed implant. In some embodiments, actuation of a cam twist grip of the inserter portion may spread the rotating jaw and non-rotating jaw apart from one another in a horizontal direction allowing for alignment and engagement with the gripping protrusions. In some embodiments, a rotating jaw may engage with a gripping protrusion of the superior endplate to move the implant to an expanded position. In some embodiments, the disclosed surgical tool may include a drive portion configured for insertion in the surgical tool and extending longitudinally in a proximal to distal direction of the inserter portion to expand and lock the expandable implant. In various embodiments, the actuation of the rotation knob of the drive portion may spread the rotating jaw and non-rotating jaw apart from one another in a vertical direction thereby pivotally expanding the expandable implant. In various embodiments, the surgical tool may include at least one angle indicator to identify respective angle of inclination positions of the implant. In at least some embodiments, a plurality of angle indicators may be provided that include respective rotatable dials.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a perspective view of an expandable implant.

FIG. 2 is an alternate perspective view of an expandable implant.

FIG. 3 is a top down view of an expandable implant.

FIG. 4 is a side view of an expandable implant.

FIG. 5 is a rear perspective view of an expandable implant.

FIG. 6 is a perspective view of the interior of a superior endplate of an expandable implant.

FIG. 7 is a perspective view of the interior of an inferior endplate of an expandable implant.

FIG. 8 is a perspective exploded parts view of an expandable implant.

FIG. 9 an exploded parts view of an expandable implant from a side view perspective.

FIG. 10 is a perspective cross section view of an expandable implant.

FIG. 11 is a cross section view of an expandable implant.

FIG. 12 is a side view of a superior endplate for use with at least some expandable implant embodiments.

FIG. 13A is a perspective view of a first expandable implant.

FIG. 13B is a perspective view of a second expandable implant.

FIG. 14A is a perspective view of a third expandable implant.

FIG. 14B is a perspective view of a fourth expandable implant.

FIG. 15 is a perspective view of a fifth expandable implant.

FIG. 16 is a side view of an expandable implant in the expanded configuration.

FIG. 17 is a side view of an expandable implant showing a bone screw trajectory.

FIG. 18 is a front view of an expandable implant.

FIG. 19 is a front view of an enlarged area of FIG. 18.

FIG. 20 is a perspective view of an inserter for use with disclosed expandable implants.

FIG. 21 is a perspective view of an inserter for use with disclosed expandable implants shown in skeleton outlining for ease of understanding.

FIG. 22 is a perspective view of an inserter for use with disclosed expandable implants shown in skeleton outlining for ease of understanding.

FIG. 23A is a rear view of an inserter in a non-expanded position.

FIG. 23B is a rear view of an inserter in an expanded position.

FIG. 24 is an enlarged view of a distal end of an inserter in an expanded position coupled to an example expandable implant in a corresponding expanded position.

FIG. 25 is a perspective view of an expandable implant in an expanded configuration after a breakoff portion of a locking screw has been broken off.

FIG. 26 is a perspective view of a surgical instrument for use with disclosed expandable implants.

FIG. 27 is a perspective view of a surgical instrument for use with disclosed expandable implants.

FIG. 28 is a perspective view of an expandable implant after the mounting tangs have been broken off.

FIG. 29 is a reference drawing showing the human spine of which various disclosed implant embodiments may be installed in.

FIG. 30 is a reference drawing showing various planes and reference directions of which the various disclosed implant embodiments may move in or act in with respect to a patient.

FIG. 31 is a perspective view of a second implant embodiment.

FIG. 32 is a first perspective exploded parts view of the second implant embodiment.

FIG. 33 is a second perspective exploded parts view of the second implant embodiment.

FIG. 34 is a side exploded parts view of the second implant embodiment.

FIG. 35 is a first side view of a breakoff screw having a recessed fracture surface.

FIG. 36 is a second side view of a breakoff screw having a recessed fracture surface.

FIG. 37 is a cross section view of a breakoff screw having a recessed fracture surface.

FIG. 38 is a perspective view of a swaging fixture.

FIG. 39 is a cross section view of a swage mandrel and a distal end of a breakoff screw before the commencement of a swage process.

FIG. 40 is a cross section view showing a result of a swage process.

FIG. 41 is an enlarged view of region S-W of FIG. 40.

FIG. 42A is a front perspective view of a third implant embodiment.

FIG. 42B is an alternate front perspective view of a third implant embodiment.

FIG. 43A is a front perspective view of an implant having angled engagement features.

FIG. 43B is a top down view of an implant having angled engagement features.

FIG. 44 is a perspective view of a surgical tool for use with disclosed implant embodiments.

FIG. 45 is a first side view of a surgical tool for use with disclosed implant embodiments.

FIG. 46A is a second side view of a surgical tool for use with disclosed implant embodiments.

FIG. 46B is a third side view of a surgical tool in an operative position for use with disclosed implant embodiments.

FIG. 46C is a perspective view of a surgical tool in the operative position.

FIG. 47A is first exploded parts view of a surgical tool for use with disclosed implant embodiments.

FIG. 47B is second exploded parts view of a surgical tool for use with disclosed implant embodiments.

FIG. 48 is a perspective view showing a surgical tool immediately prior to coupling with a surgical implant.

FIG. 49 is a first cross section drawing of the surgical tool and implant in a coupled configuration.

FIG. 50 is a second cross section drawing of the surgical tool and implant in a coupled configuration.

FIG. 51 is a third cross section drawing of the surgical tool and implant in a coupled configuration.

FIG. 52 is a perspective view showing a parallel jaw inserter and a drive portion in an assembled configuration.

FIG. 53 is a perspective view showing a parallel jaw inserter with an outer body assembly and a drive portion disassembled.

FIG. 54 is an exploded parts view of a parallel jaw inserter and a drive portion for use with disclosed implant embodiments.

FIG. 55 is a perspective view of a distal end of a parallel jaw inserter.

FIG. 56 is a perspective exploded parts view of a distal end of a parallel jaw inserter.

FIG. 57 is a top-down perspective view showing exploded parts view of a distal end of a parallel jaw inserter.

FIG. 58 is a second perspective exploded parts view of a distal end of a parallel jaw inserter.

FIG. 59 is a first perspective view showing a parallel jaw inserter without an outer body assembly.

FIG. 60 is a second perspective view showing a parallel jaw inserter without an outer body assembly.

FIG. 61 is a perspective view showing a distal end of a parallel jaw inserter with parallel jaw closed and rotating jaw down.

FIG. 62 is a perspective view showing a distal end of a parallel jaw inserter with parallel jaw closed and rotating jaw up.

FIG. 63 is a perspective view showing a distal end of a parallel jaw inserter with parallel jaw open and rotating jaw down.

FIG. 64 is a perspective view showing a distal end of a parallel jaw inserter with parallel jaw open and rotating jaw up.

FIG. 65A is a perspective view showing a proximal end of a drive portion.

FIG. 65B is a perspective view showing a distal end of a parallel jaw inserter.

### DETAILED DESCRIPTION

Embodiments of the present disclosure relate generally, for example, to spinal stabilization systems, and more particularly, to surgical instruments for use with spinal stabilization systems. Embodiments of the devices and methods are described below with reference to the Figures.

The following discussion omits or only briefly describes certain components, features and functionality related to medical implants, installation tools, and associated surgical techniques, which are apparent to those of ordinary skill in the art. It is noted that various embodiments are described in detail with reference to the drawings, in which like reference numerals represent like parts and assemblies throughout the several views, where possible. Reference to various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Terms such as “same,” “equal,” “planar,” “coplanar,” “parallel,” “perpendicular,” etc. as used herein are intended to encompass a meaning of exactly the same while also including variations that may occur, for example, due to manufacturing processes. The term “substantially” may be used herein to emphasize this meaning, particularly when the described embodiment has the same or nearly the same functionality or characteristic, unless the context or other statements clearly indicate otherwise. The term “about” may also be used herein to emphasize this meaning and if a value and/or a range of values is provided in the specification or claims with the modifier “about” a meaning of +/−ten percent (10%) to those provided values are encompassed by the meaning of “about,” unless the context clearly indicates otherwise.

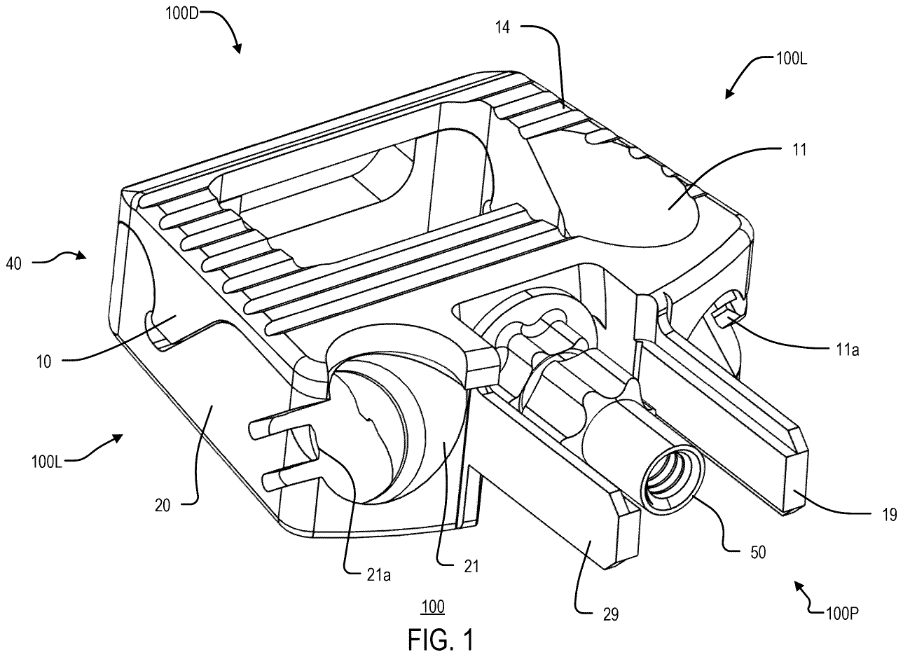

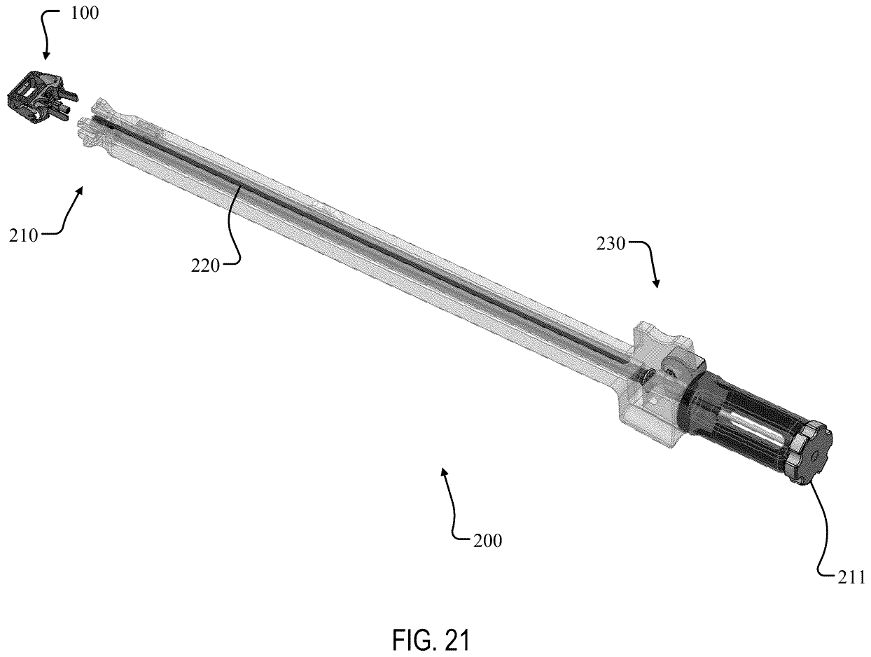

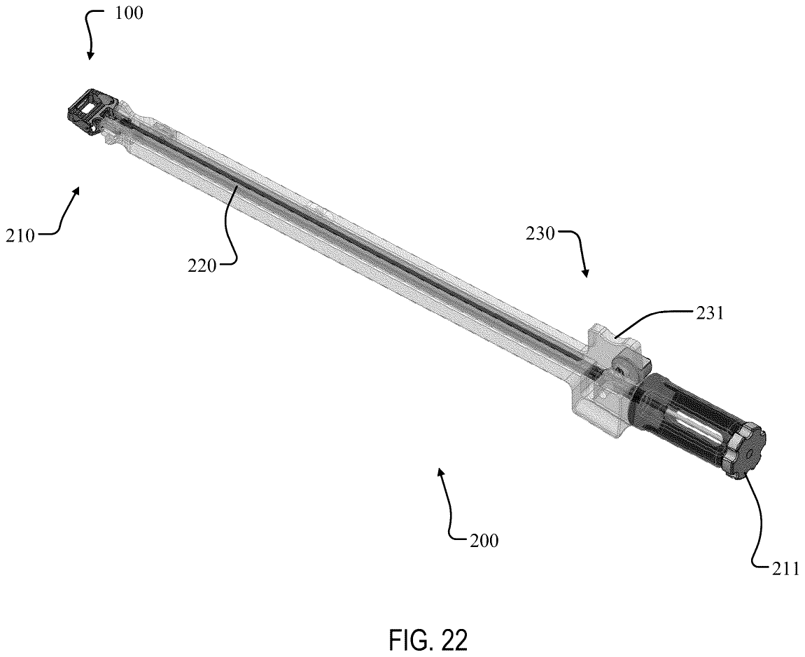

Referring to FIGS. 1-43 generally, various embodiments and views of an expandable implant 100 are disclosed. The components of expandable implant 100 can be fabricated from biologically acceptable materials suitable for medical applications, including metals, synthetic polymers, ceramics and bone material and/or their composites. For example, the components, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL™), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe, polylactic acid or polylactide and their combinations.

In various embodiments, components may be coated with a ceramic, titanium, and/or other biocompatible material to provide surface texturing at (a) the macro scale, (b) the micro scale, and/or (c) the nano scale, for example. Similarly, components may undergo a subtractive manufacturing process providing for surface texturing configured to facilitate osseointegration and cellular attachment and osteoblast maturation. Example surface texturing of additive and subtractive manufacturing processes may comprise (a) macro-scale structural features having a maximum peak-to-valley height of about 40 microns to about 500 microns, (b) micro-scale structural features having a maximum peak-to-valley height of about 2 microns to about 40 microns, and/or (c) nano-scale structural features having a maximum peak-to-valley height of about 0.05 microns to about 5 microns. In various embodiments, the three types of structural features may be overlapping with one another, for example. Additionally, such surface texturing may be applied to any surface, e.g., both external exposed facing surfaces of components and internal non exposed surfaces of components. Further discussion regarding relevant surface texturing and coatings is described in, for example, U.S. Pat. No. 11,096,796, titled Interbody spinal implant having a roughened surface topography on one or more internal surfaces, and filed on March, 4, 2013—the entire disclosure of which is incorporated herein by reference in its entirety. Accordingly, it shall be understood that any of the described coating and texturing processes of U.S. Pat. No. 11,096,796, may be applied to any component of the various embodiments disclosed herein, e.g., the exposed surfaces and internal surfaces of endplates. Another example technique for manufacturing an orthopedic implant having surfaces with osteoinducting roughness features including micro-scale structures and nano-scale structures is disclosed in U.S. Pat. No. 10,821,000, the entire contents of which are incorporated herein by reference. Additionally, an example of a commercially available product may be the Adaptix™ Interbody System sold by Medtronic Spine and comprising a titanium cage made with Titan nanoLOCK™.

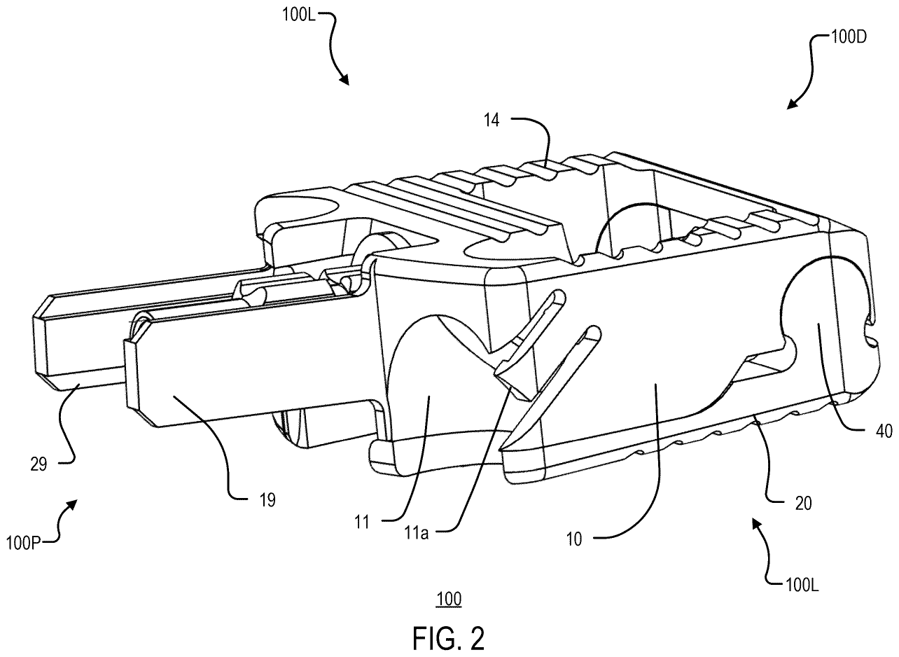

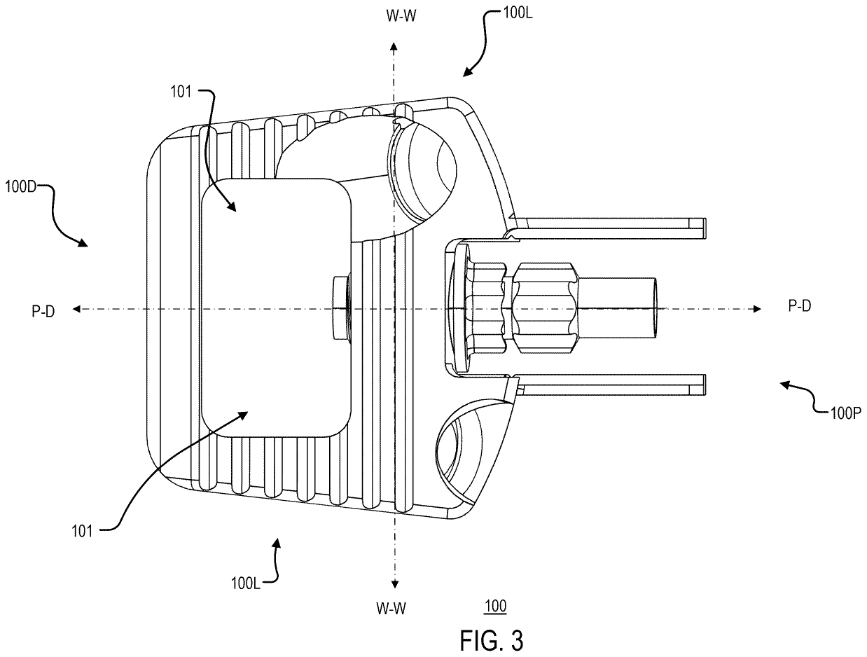

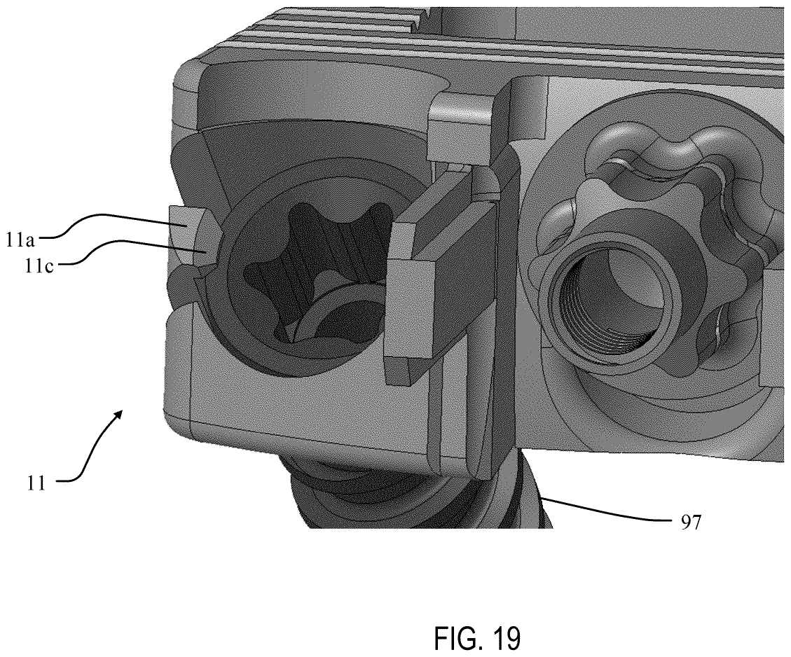

Referring generally to FIGS. 1-5 various views of an expandable implant 100 in a collapsed position are illustrated. FIGS. 1-2 are various perspective views of an expandable implant 100. FIG. 3 is a top down view of an expandable implant 100. In the example embodiment, expandable implant 100 may include a proximal end 100P, a distal end 100D, and first and second lateral sides 100L. Additionally, a pair of bone screw apertures 11, 21 may be positioned on the proximal end 100P, for example. In various embodiments, bone screw apertures 11, 21 may comprise a corresponding bone screw retention mechanism 11a, 21a (may also be referred to as an anti-backout locking mechanism). In the example embodiment, the bone screw retention mechanisms 11a, 21a, comprise a flexible tang member having a hook portion at an end thereof that allows the flexible tang member to flex outward in a lateral direction away from the corresponding bone screw aperture 11, 21 during initial installation of the bone screw and to flex back inward towards the corresponding bone screw aperture 11, 21 to prevent a corresponding bone screw from backing out. For example, as the bone is installed in bone screw aperture 11, 21, the bone screw retention mechanism 11a, 21a, may flex outward as the underside of the head portion contacts inclined surface 11c (see FIG. 19).

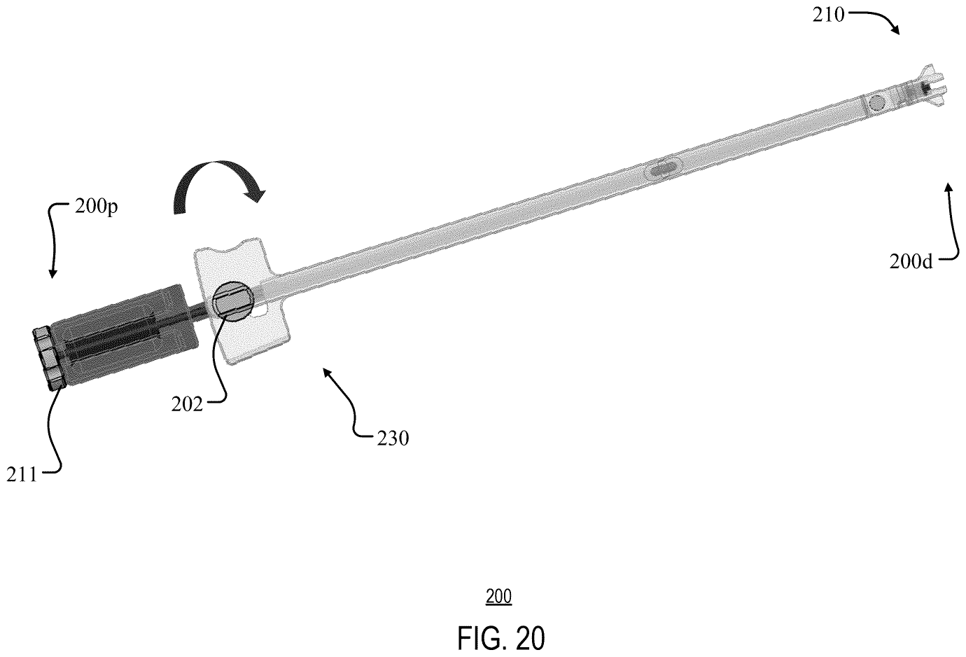

In various embodiments, and as illustrated in FIGS. 1-2, mounting tangs 19, 29 may extend in a proximal direction (may also be referred to as an anterior direction depending on surgical technique and orientation), for example. In various embodiments, implant 100 may be referred to as an externally driven expandable implant because an end user or surgeon may use a surgical tool to open and close implant 100, e.g. expand implant 100. For example, an external tool may adjust the lordotic angle of implant 100 as will be explained in detail with respect to FIGS. 20-25. Once implant 100 is expanded to an appropriate lordotic angle (also referred to as angle of inclination), an end user may fix the relative angle of the superior endplate 10 relative to the inferior endplate 20 by tightening locking screw 50, for example. In some embodiments, superior endplate 10 may be referred to as a “cephalad” endplate and inferior endplate 20 may be referred to as a “caudal” endplate.

Locking screw 50 may also be used in other embodiments, such as fixation of posterior rods, fixation of pedicle screws, and other set screw constructs. Additionally, locking screw 50 may be referred to as a “breakoff screw” in some embodiments.

At least one advantage of relying on an external tool to adjust a lordotic angle of implant 100 may be the reduction of internal components within implant 100 relative to other forms of implants relying on various moving mechanisms and/or expansion mechanisms, for example. Accordingly, in various embodiments, implant 100 may have a relatively large void space in the interior thereof, which may facilitate a fusion process during an ACDF procedure. For example, implant 100 may have a relatively large internal volume 101 that is open through the superior endplate 10 and inferior endplate 20 which may be packed with bone graft material, for example.

As illustrated in FIG. 3, implant 100 may extend in a proximal-to-distal direction (may also referred to as a longitudinal direction and/or an anterior-to-posterior direction depending on surgical technique and final orientation) from the proximal end 100P (may be referred to as anterior end depending on surgical technique and final orientation) to the distal end 100D (may be referred to as posterior end depending on surgical technique and final orientation) though axis P-D through the center of the implant 100, for example. Implant 100 may extend in a widthwise direction (also referred to as lateral direction) from the first lateral side 100L to the second lateral side 100L through axis W-W through the center of the implant 100 and the center of locking screw 50, for example. The axis P-D may be perpendicular and/or substantially perpendicular to the axis W-W. For example, the proximal-to-distal direction may be perpendicular to the widthwise direction. Additionally, a width of the implant may taper from a proximal end 100P where it is widest towards a distal end 100D where it is narrowest. In various embodiments, implant 100 may extend from a superior endplate 10 to an inferior endplate 20 in a heightwise direction (may also be referred to as a cephalad-to-caudal and/or vertical direction depending on surgical technique and final orientation).

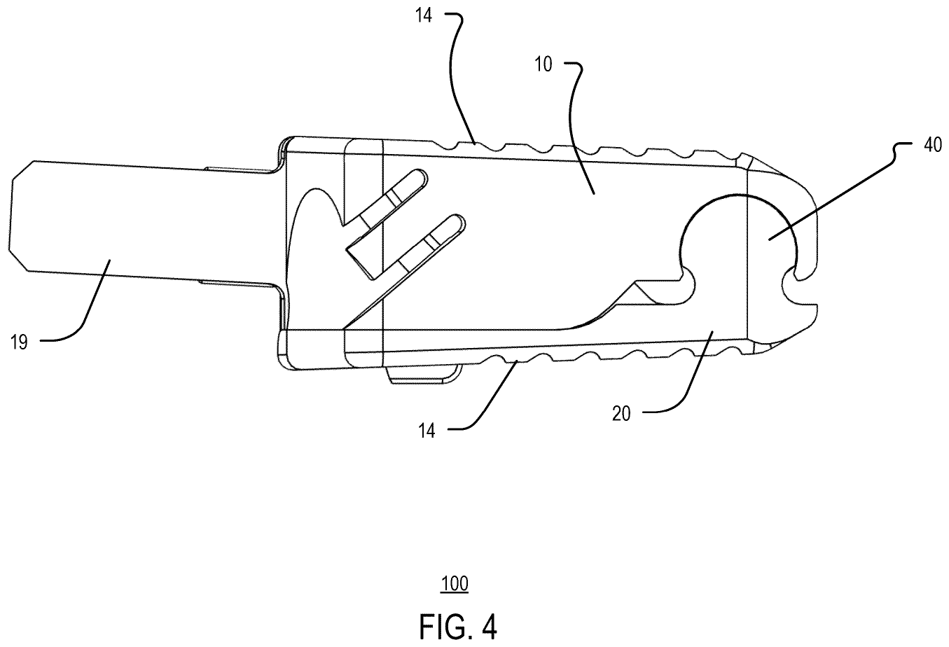

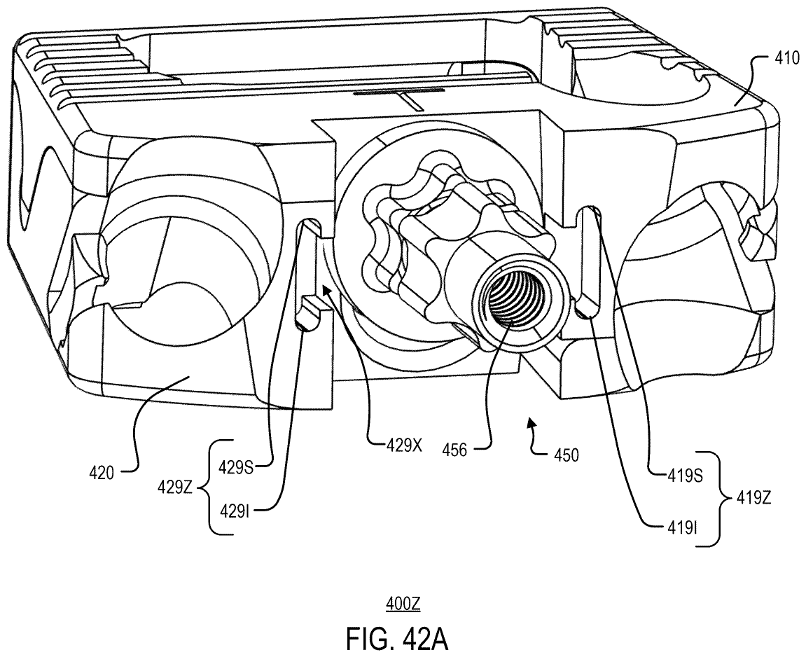

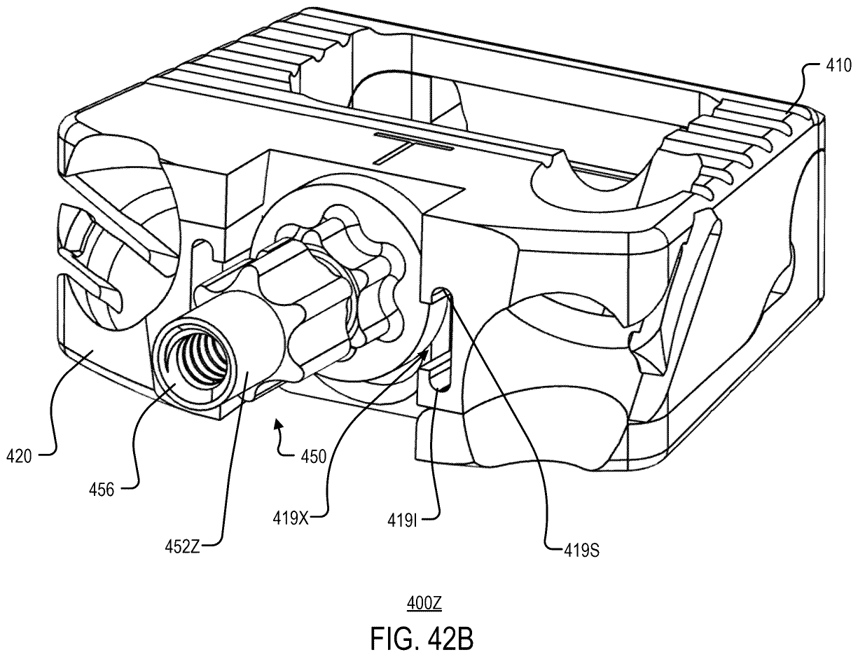

FIG. 4 is a side view of an expandable implant 100. In the example illustration, it is shown that a superior endplate 10 is connected to an inferior endplate 20 such that the superior endplate may pivot about a hinge member 40. In the example embodiment, hinge member 40 comprises an arcuate rail portion of inferior endplate 20 that extends in the widthwise direction, for example. In other embodiments, the hinge member 40 may be reversed relative to the superior and inferior endplates than as illustrated in FIG. 4. In the example embodiment, hinge member 40 may be nested into a corresponding arcuate cavity of the superior endplate 10 such that superior endplate 10 may expand and/or otherwise rotate about hinge member 40. Additionally, in various embodiments the superior endplate 10 and/or inferior endplate 20 may include various engagement elements 14 for engaging with an adjacent boney structure such as a vertebrae, for example. In the example embodiment, the engagement elements comprise a series of alternating rails and valleys therebetween that extend in the widthwise direction. In some embodiments, the valleys may be angled about 20-40 degrees and in at least one embodiment the valleys may be angled at about 30 degrees relative to the corresponding rail (see FIGS. 42A and 42B). At least one advantage of this orientation may be a relatively greater resistance and/or suppression of expulsion of the implant 100 in both the lateral direction and in the proximal-to-distal direction. However, claws, hooks, dimples, spikes, etc. are also contemplated as example engagement elements 14. In some embodiments, an acid etching process may be utilized to form a roughened or textured surface to facilitate securing the implant between boney portions and/or suppressing expulsion of implant 100.

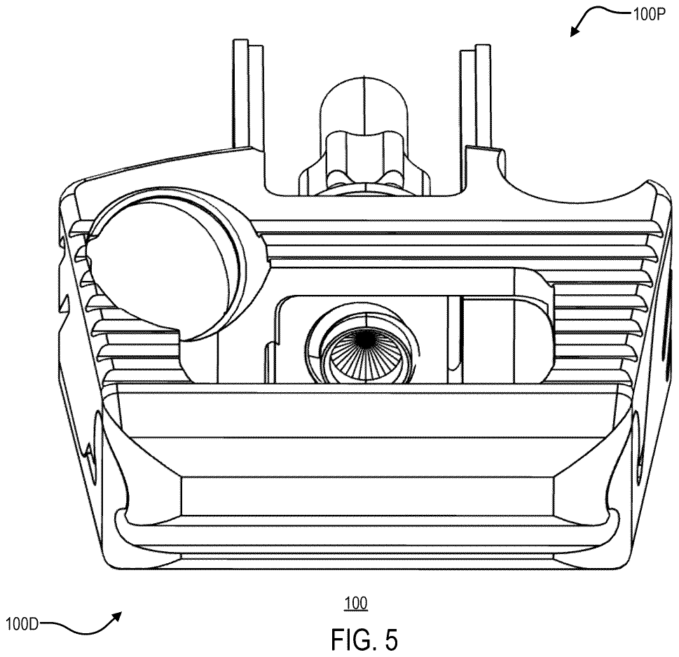

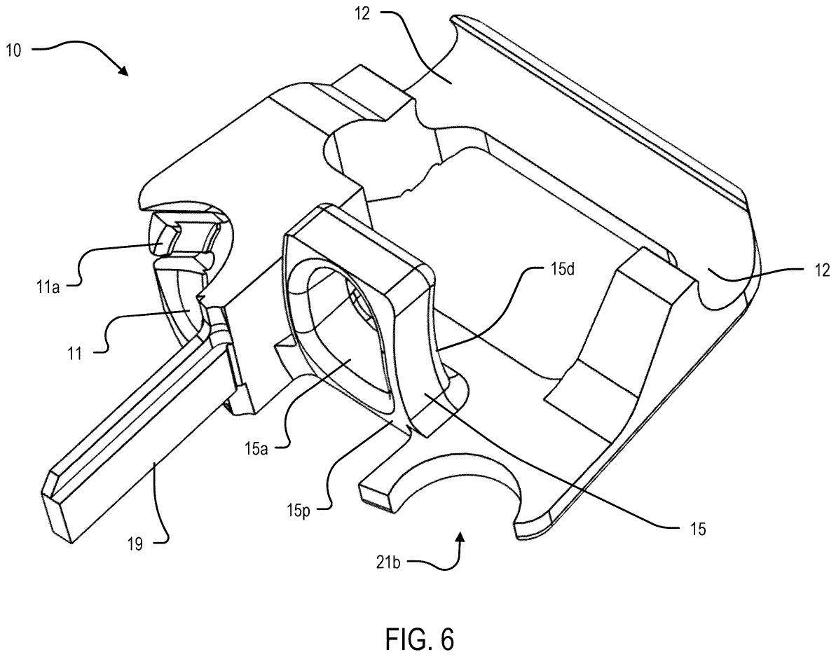

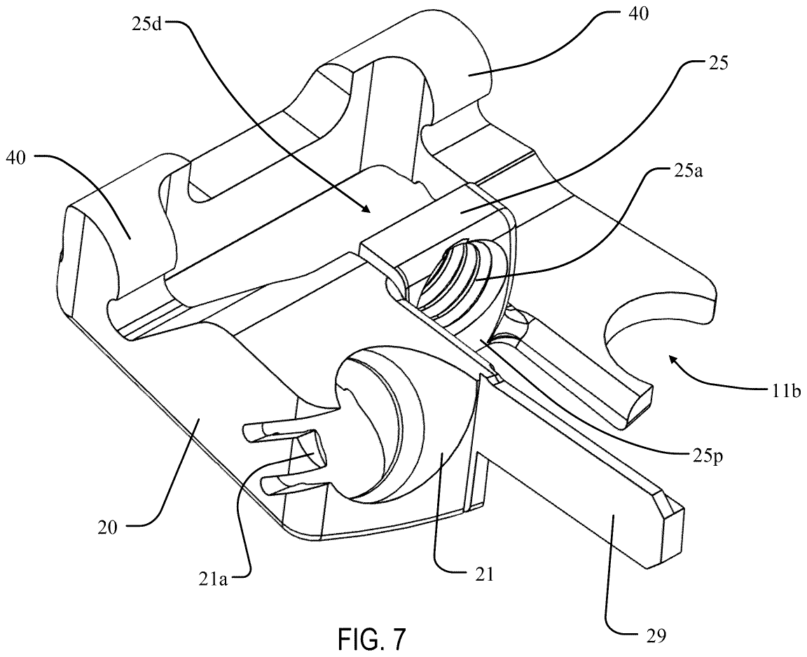

FIG. 5 is a rear perspective view of an expandable implant 100. In the example illustration it is shown that the distal end 100D is narrower than the proximal end 100P. FIG. 6 is a perspective view of the interior of a superior endplate 10. In the example illustration, it is shown that the distal end of superior endplate 10 includes an arcuate channel 12 of which the hinge member 40 may be disposed inside of. The proximal end of superior endplate 10 may include a bone screw aperture cutout 21b to allow a relief area for a corresponding bone screw to be insert through bone screw aperture 21 of inferior endplate 20, for example. Superior endplate 10 may also include a core 15 comprising an aperture 15a that extends from a proximal surface 15p thereof to a distal surface 15d thereof, for example. In various embodiments, aperture 15a may be referred to as a “slot”′ or “screw slot”. In some embodiments, core 15 may be referred to as a support frame and take a generally rectangular shape. In various embodiments, the distal surface 15d may be curved and generally face the distal end 100D of implant 100. FIG. 7 is a perspective view of the interior of an inferior endplate 20. In the example illustration, it is shown that the distal end of inferior endplate 20 includes a hinge member 40 in the form of an arcuate rail that may be disposed inside of the arcuate channel 12 of the superior endplate 10, for example. The proximal end of inferior endplate 20 may include a bone screw aperture cutout 11b to allow a relief area for a corresponding bone screw to be insert through bone screw aperture 11 of superior endplate 10, for example. Inferior endplate 20 may also include a core 25 comprising a threaded aperture 25a that extends from a proximal surface 25p thereof to a distal surface 25d thereof, for example. In some embodiments, core 25 may be referred to as a support frame and take a generally rectangular shape. In various embodiments, the superior endplate 10 and inferior endplate 20 may each be formed by a unitary single piece, respectively.

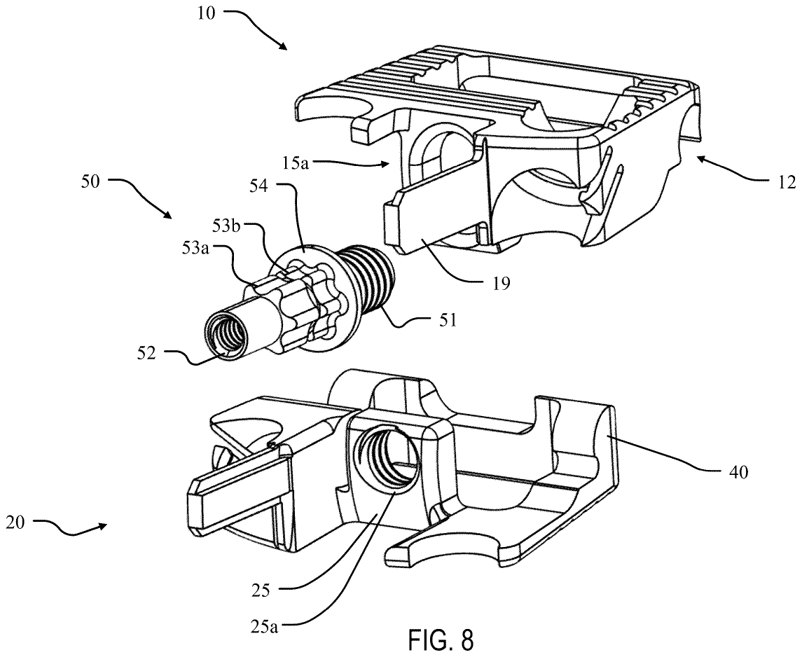

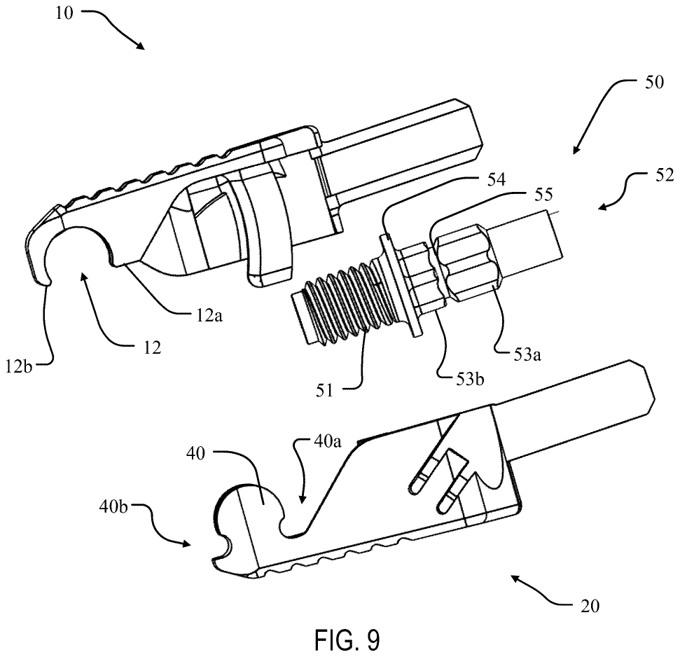

FIG. 8 is a perspective exploded parts view and FIG. 9 is an exploded parts view from a side view perspective of an expandable implant 100. In the example embodiment, a locking screw 50 is illustrated. Locking screw 50 may include an external thread pattern 51 on an outside circumferential surface thereof, for example. The external thread pattern 51 of locking screw 50 may have a size and shape generally corresponding to the threaded aperture 25a of core 25 of inferior endplate 20, for example. In various embodiments, an engagement surface 54 may be disposed adjacent and proximal of external thread pattern 51. In the example embodiment, engagement surface 54 is shaped like a washer and is directly connected to locking screw 50. However, in other embodiments, engagement surface 54 may be a washer or separated element, for example. In some embodiments, engagement surface 54 may be conically shaped and/or spherically shaped. Engagement surface 54 may include a relatively planar and/or flat distal surface and/or proximal surface (anterior/ventral surface). In various embodiments, a proximal end of set screw 50 may include an aperture having an internal threaded surface 52. For example, a cylindrical shaped proximal end may include an aperture having a thread pattern disposed on an internal circumferential surface of the cylindrical shaped proximal end. In the example embodiment, a first drive feature 53a and a second drive feature 53b may be disposed adjacent to and distally with respect to a proximal most end of set screw 50. Additionally, first and second drive features 53a, 53b may be disposed proximally with respect to engagement surface 54. In the example embodiment, drive features 53a, 53b take a hexalobular shape, although various other shapes such as hexagonal, polygonal, Torx, etc. are also contemplated. In some embodiments, a surgical drive tool having a corresponding socket may be coupled to drive features 53a, 53b to cause rotation of locking screw 50. Similarly, in some alternative embodiments, a drive tool with a protruding threaded member having a thread pattern with a corresponding size and shape to internal threaded surface 52 may also cause rotation of set screw 50.

As seen best in FIG. 9, set screw 50 may also include a breakoff location 55, for example. In the example embodiment, breakoff location 55 is disposed directly between drive features 53a, 53b and is designed to shear off when a sufficient rotational force (torque) is applied to a proximal end of set screw 50 while a distal end of set screw 50 is stationary, e.g., when set screw 50 is secured in a locked position and a continued rotational force (torque) is applied to the proximal end of set screw 50 the drive feature 53a and cylindrical end having the internal threaded surface 52 may breakoff. In various embodiments, the internal threaded surface 52 may also be utilized to ensure that the broken off portion is removed from the patient and remains connected to a surgical tool/breakoff tool. As also seen best in FIG. 9, the inferior endplate 20 may include a first relief 40a and a second relief 40b on opposite sides of hinge member 40. The first relief 40a may have a size and shape corresponding to a size and shape of a first portion 12a of superior endplate 10 and the second relief 40b may have a size and shape corresponding to a size and shape of a second portion 12b of superior endplate 10, for example. In various embodiments, portions 12a, 12b may comprise a hook shape, outdent, and/or protrusion, for example. In the example embodiment, portions 12a, 12b may be disposed on opposite sides of channel 12 and cup hinge member 40 such that the superior endplate 10 and inferior endplate 20 may rotate relative to one another without becoming uncoupled.

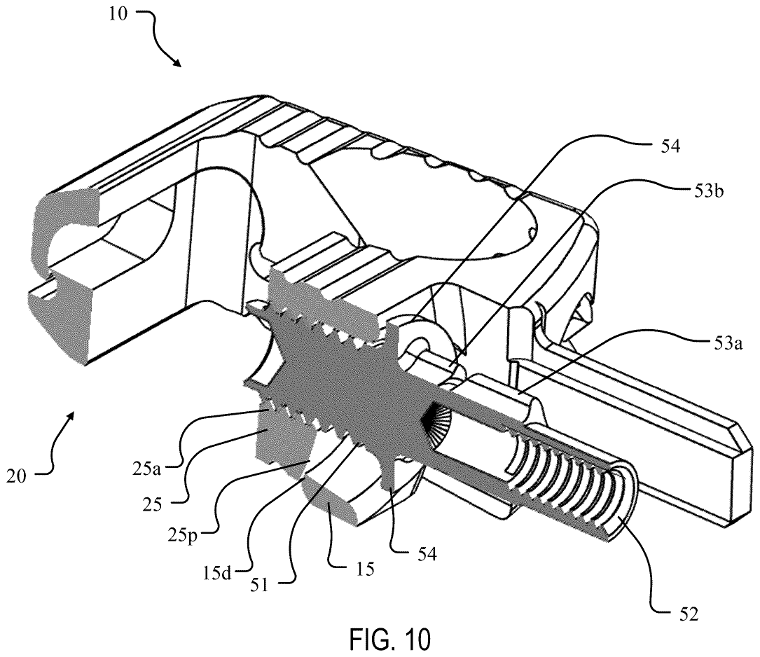

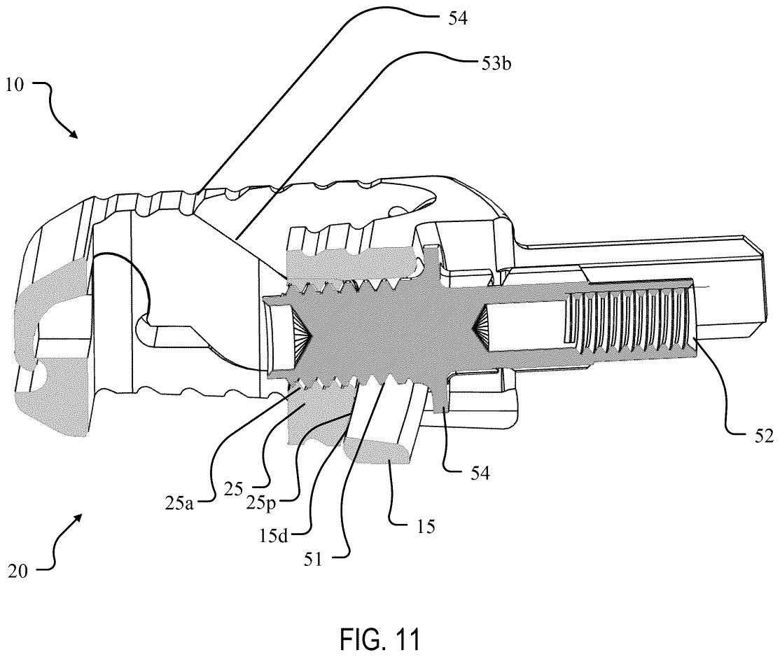

FIG. 10 is a perspective cross section view and FIG. 11 is a cross section view of expandable implant 100. In the example embodiment, the superior endplate 10 and inferior endplate 20 are coupled together by hinge member 40, and core 25 may be positioned behind of core 15, e.g., core 25 may be positioned distally with respect to core 15. Additionally, the outside external thread pattern 51 of locking screw 50 may engage with the threaded aperture 25a of the core 25 and extend through aperture 15a of core 15. In this way, when locking screw 50 is rotated, the distal surface 15d of core 15 may engage with the proximal surface 25p of core 25. For example, by tightening locking screw 50 the engagement surface 54 of locking screw 50 pushes against the proximal surface 15p of core 15 thereby bringing the superior endplate 10 and inferior endplate 20 into frictional engagement.

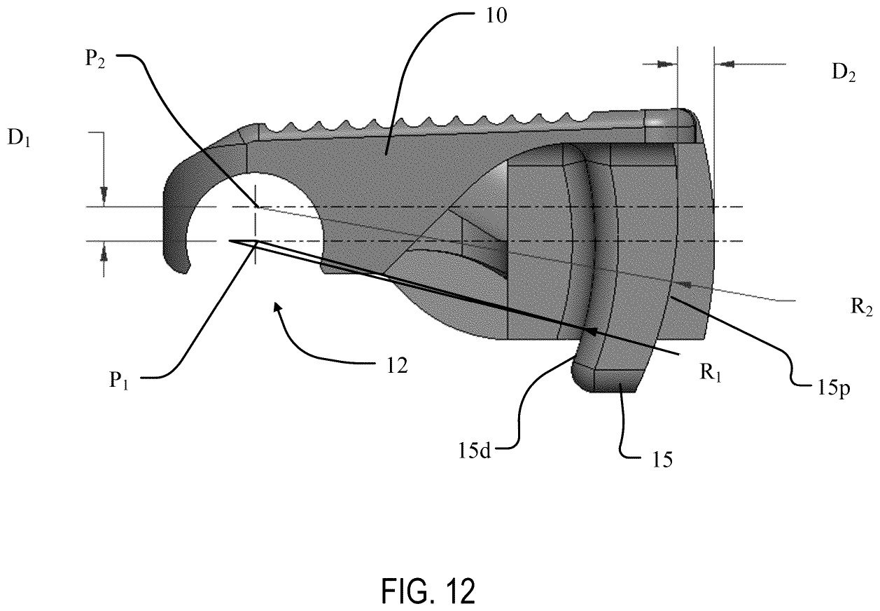

FIG. 12 is a side view of a superior endplate 10 for use with at least some expandable implant 100 embodiments. In the example embodiment, superior endplate 10 may include an arcuate channel 12 of which the hinge member 40 may be disposed inside of. In various embodiments, arcuate channel 12 may be defined by a first circle having a center point at P₁ and/or a segment of the circle having the center point at P₁. The center point P₁ may define an axis of rotation that superior endplate 10 may rotate and/or pivot with respect to inferior endplate 20. For example, superior endplate 10 may be hingedly coupled to hinge member 40 as explained above and rotatable about an axis of rotation defined by center point P₁, for example. Additionally, in various embodiments a distal surface 15d of core 15 may be a curved surface defined (in part or in total) by a second circle having a center point at P₁ and a radius R₁. The proximal surface 15p of core 15 may also be a curved surface defined (in part or in total) by a segment of a circle having a radius R₂ and a center point P₂. In the example embodiment, P₂ is located a distance D₁ above point P₁ and radius R₂ is greater than radius R₁. Additionally, the proximal surface 15p of core 15 is offset a distance D₂ from the proximal most face of the superior endplate 10. In the example embodiment, center point P₂ is vertically above center point P₁ however, in other embodiments, center point P₂ may be offset by a greater amount or even a lesser amount than illustrated. In some examples, P₂ may not be aligned vertically above P₁. In various embodiments, R₁ may be about 7-9 mm +/−about 1 mm and R₂ may be about 8-10 mm +/−about 1 mm although these numbers may be modified in some embodiments having a larger or smaller footprint. In various embodiments D₁ is about 0.25 mm to about 1.0 mm and D₂ is about 0.25 mm to about 1.25 mm. In at least one embodiment, D₁ is about 0.75 mm and D₂ is about 0.8 mm and R₂ is about 9.2 mm.

The above explained geometrical relationship between the offset center points P₁ and P₂ and R₁ and R₂ may have several advantages in terms of operability and functionality. At least one advantage is that the superior endplate 10 may have a natural tendency to apply a force against the engagement surface 54 of locking screw 50 such that locking screw 50 may function similar to a wedge preventing implant 100 from fully collapsing. Another advantage is that a biasing force may be applied that naturally urges the superior endplate 10 and inferior endplate 20 into an expanded position which may assist with expanding the implant 100 when positioned between a superior vertebrae and an inferior vertebrae, for example. For example still, an end user such as a surgeon may expand implant 100 and the offset arrangement explained above may facilitate the function of keeping implant 100 lordosed at the chosen angle.

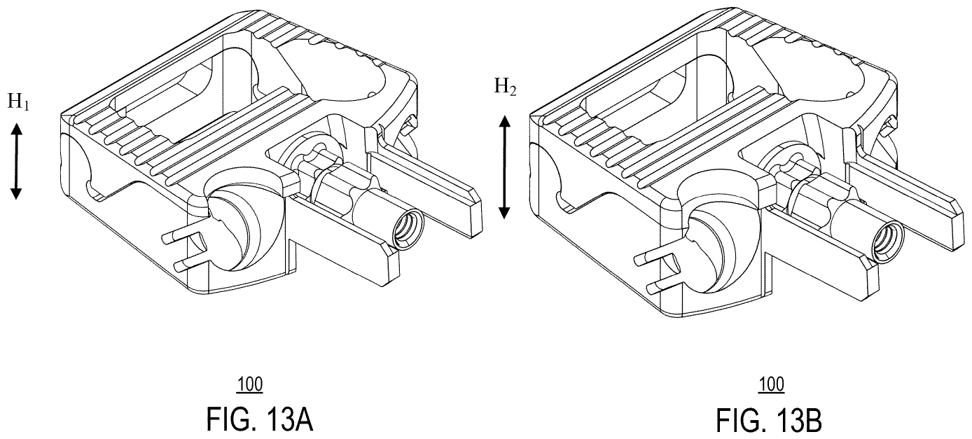

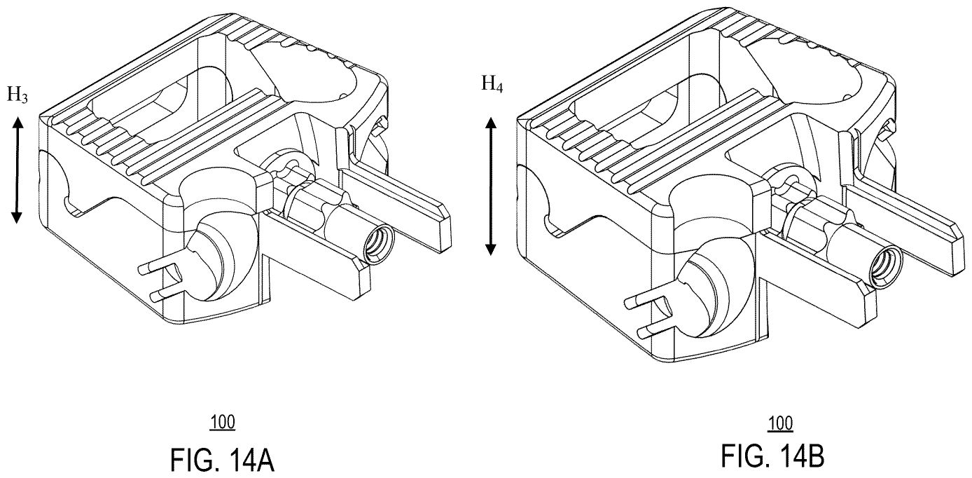

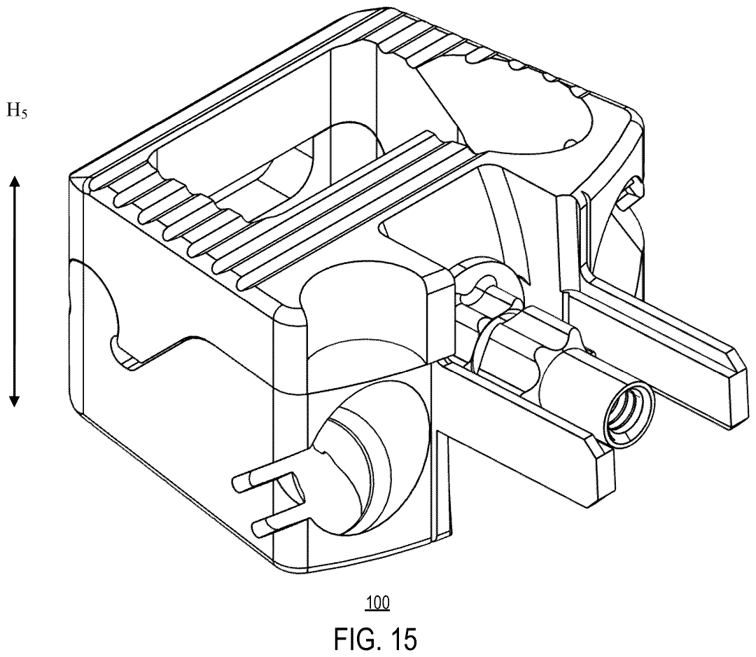

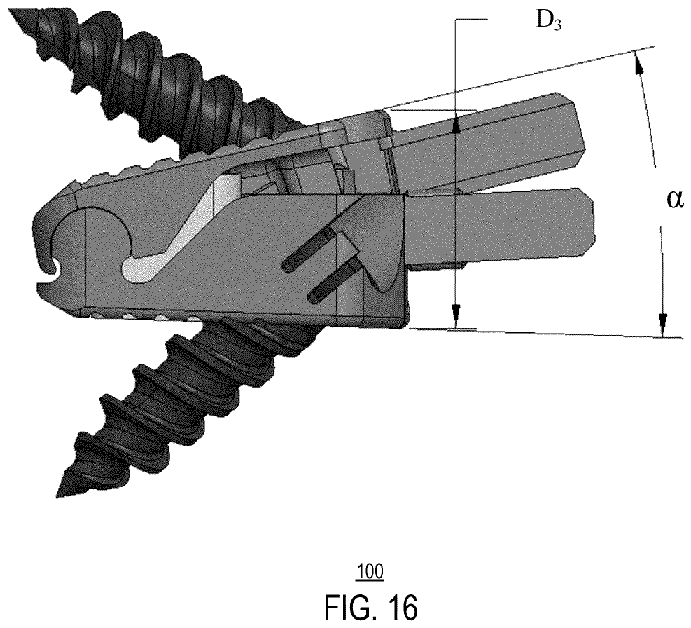

FIG. 13A is a perspective view of a first expandable implant, FIG. 13B is a perspective view of a second expandable implant, FIG. 14A is a perspective view of a third expandable implant, FIG. 14B is a perspective view of a fourth expandable implant, and FIG. 15 is a perspective view of a fifth expandable implant. In the series of illustrations it is shown that various embodiments in accordance with the principles of this disclosure may be variously sized depending on the particular location in a human body and the particular patient specific human anatomy. For example, FIG. 13A illustrates a first expandable implant 100 having a first height H₁ or thickness between the superior endplate 10 and inferior endplate 20, FIG. 13B illustrates a second expandable implant 100 having a second height H₂ or thickness, FIG. 14A illustrates a third expandable implant 100 having a third height H₃ or thickness, FIG. 14B illustrates a fourth expandable implant 100 having a fourth height H₄ or thickness, and FIG. 15 illustrates a fifth expandable implant 100 having a fifth height H₅ or thickness. In at least some embodiments, H₁ may be about 5 mm, H₂ may be about 6 mm, H₃ may be about 7 mm, H₄ may be about 8 mm, H₅ may about 9 mm, for example. In various embodiments, an angle of inclination between the superior endplate 10 and inferior endplate 20 may be about 4 degrees to about 15 degrees in an expanded configuration, e.g., an angled and/or inclined configuration.

FIG. 16 is a side view of an expandable implant 100 in the expanded configuration. In an expanded position, a distance D₃ between the superior endplate 10 and inferior endplate 20 at the proximal end 100P may be relatively greater than in the closed configuration, for example. Additionally, an angle of inclination α may be relatively greater in an expanded position than in the closed configuration, for example. In this embodiment, implant 100 may have a height H₁ corresponding to FIG. 13A and be about 5 mm in a closed configuration. In the illustrated expanded configuration of FIG. 16, D₃ may be about 8 mm to 9 mm and α may be about 10 degrees to about 20 degrees. In at least one embodiment, D₃ may be 8 mm in a fully expanded position and α may be about 15 degrees, for example.

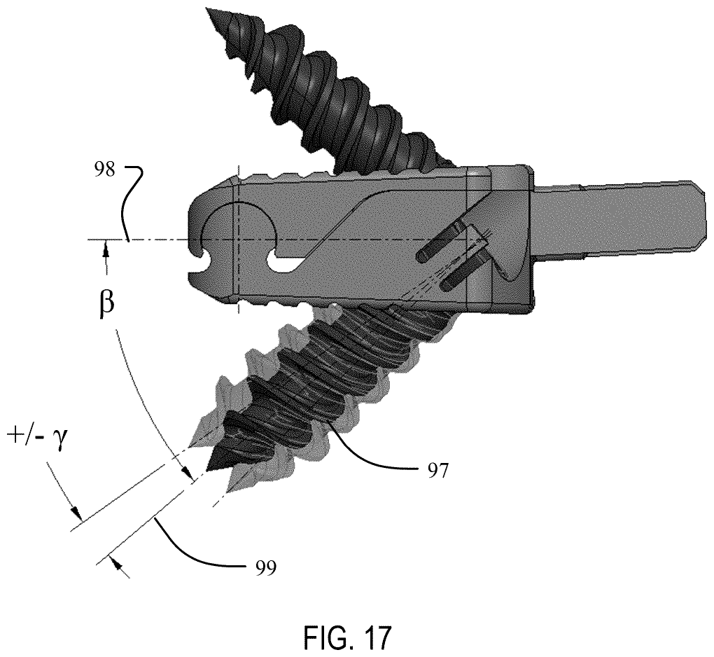

FIG. 17 is a side view of an expandable implant 100 showing a bone screw trajectory 99. In the example embodiment, it is shown that a centered bone screw trajectory 99 of bone screw 97 is at an angle β with respect to a plane 98 that crosses through a center of the implant from a first lateral side to a second lateral side, for example. Additionally, the bone screw trajectory 99 may be varied +/−by a degree γ, for example. In various embodiments, β may be about 30 degrees to about 50 degrees and γ may be about 2 degrees to about 10 degrees. In the example embodiment, β may be about 40 degrees and γ may be about 5 degrees.

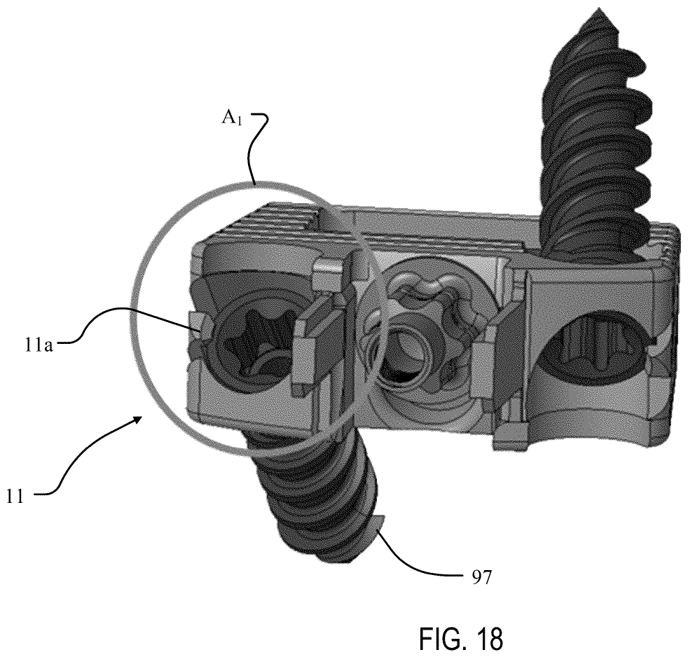

FIG. 18 is a front view of an expandable implant showing an area A₁ and FIG. 19 is a front view of an enlarged area A₁ of FIG. 18. In the example embodiment, bone screw 97 is in a position extending through bone screw aperture 11 where it cannot backout due to bone screw retention mechanism 11a. The bone screw retention mechanism 11a includes an inclined surface 11c such that when bone screw 97 is being installed, an underside of the head portion of bone screw 97 directly contacts the inclined surface 11c thereby pushing the bone screw retention mechanism 11a laterally outward and away from bone screw aperture 11, for example. Thereafter, when bone screw 97 is installed and the head portion of bone screw 97 is beneath inclined surface 11c the bone screw retention mechanism may flex back towards bone screw aperture 11 such that it will prevent bone screw 97 from backing out, e.g., a blocking surface of bone screw retention mechanism 11a may contact an upper surface of the head portion of bone screw 97. In the example embodiment, bone screw retention mechanism 11a comprises flexible arm (or spring tab) having an inclined surface 11c (or ramp) that is disposed on a lateral end of implant 100 adjacent bone screw aperture 11.

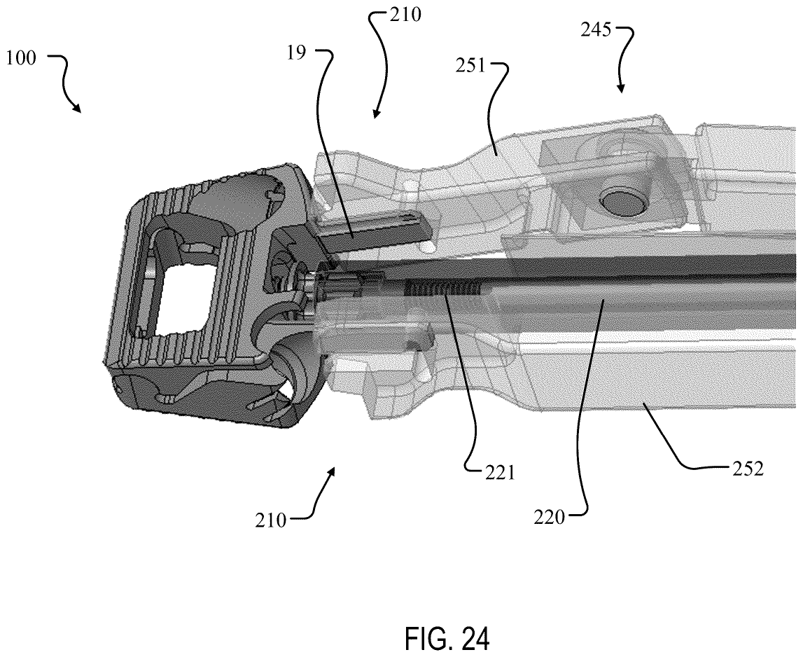

Referring generally to FIGS. 20-24 an inserter 200 for use with disclosed expandable implants 100 is illustrated. Inserter 200 may extend in a longitudinal direction from a proximal end 200p to a distal end 200d, for example. Inserter 200 may include a pair of handles 230, a handle lock 202, and mounting arms 210 for securely coupling to mounting tangs 19, 29 of implant 100, for example. Inserter 200 may further include a tightening knob 211 that is connected to a drive shaft 220 having a drive end 221. Drive end 221 may have a size and shape generally corresponding to a size and shape of the various drive features of locking screw 50, for example the internal threaded surface 52, first drive feature 53a, and/or second drive feature 53b. In the example embodiment shown in FIG. 24, drive end 221 includes an end portion having an outside threaded surface with a corresponding size and shape to the internal threaded surface 52 of locking screw 50. In various embodiments, tightening knob 211 may rotate drive shaft 220 and drive end 221 to engage drive end 221 with locking screw 50 and pull implant 100 towards inserter tool 200 such that mounting tangs 19, 29 are securely nested within corresponding channels of mounting arms 210. Additionally, an end user may rotate inserter 200 thereby translating a rotational force through drive end 221 to locking screw 50, e.g., via first drive feature 53a, and/or second drive feature 53b.

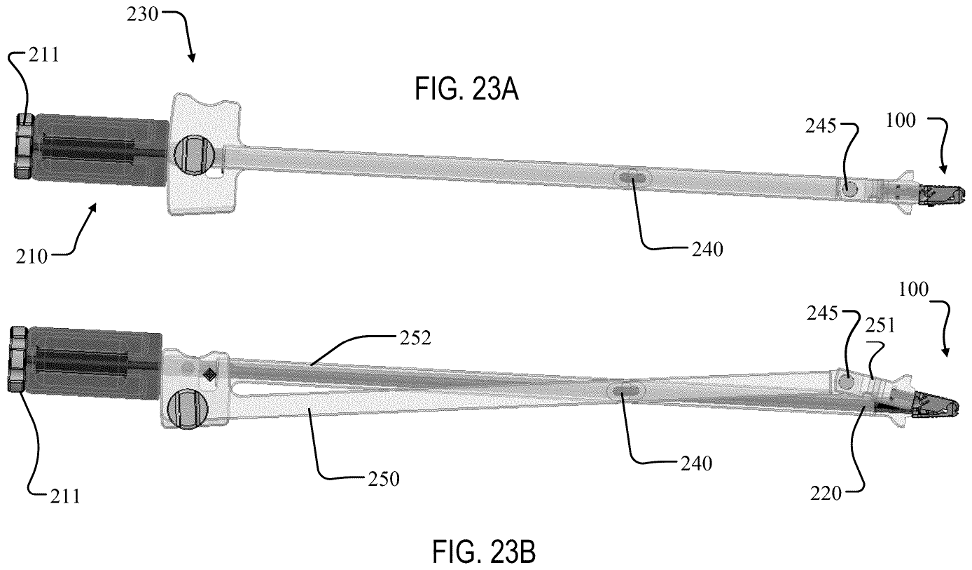

As seen best in FIGS. 23A and 23B, inserter 200 may include a pair of handles 230, a stationary arm 252, a primary pivoting arm 250, and a secondary pivoting arm 251. For example, to expand implant 100 an end user may toggle handle lock 202 to an unlocked position and push down on thumb indentation 231 of the handle 230 that is connected to the primary pivoting arm 250 and secondary pivoting arm 251. In doing so, primary pivoting arm 250 may pivot with respect to medial pivot point 240 and secondary pivoting arm 251 may pivot with respect to distal pivot point 245, for example. The path of travel of secondary pivoting arm 251 may lift up on the corresponding mounting tang 19 or 29 and cause the superior endplate 10 and inferior endplate 20 to separate from one another at the proximal end, for example. In doing so, an end user can lordose implant 100 to a desired angle. For example, as seen best in FIG. 24, secondary pivoting arm 251 has lifted up on mounting tang 19, which is nested in a corresponding channel of mounting arm 210.

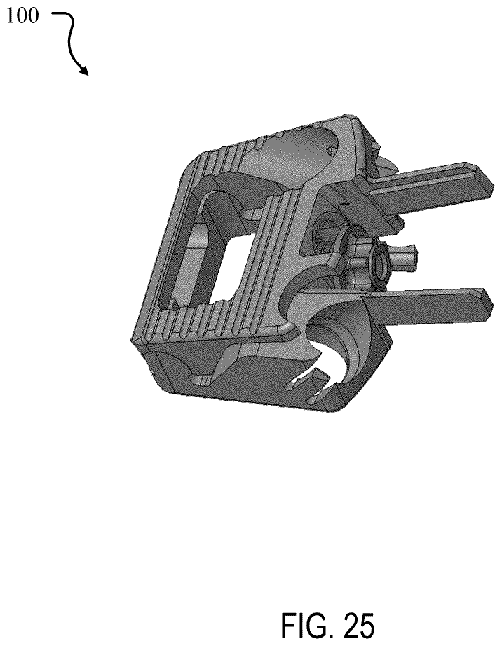

Once implant 100 is lordosed to a desired configuration, an end user may rotate drive shaft 220 and drive end 221 to tighten locking screw 50 as explained previously. After locking screw 50 is relatively tight, the end user may continue to apply a rotational force to locking screw 50 until a proximal portion comprising the cylindrical part having an internal threaded surface 52, and the first drive feature 53a breaks off at breakoff location 55. For example, once locking screw 50 is tightened to a designed torque, the locking screw 50 may shear off as explained previously. At least one advantage of utilizing the locking screw 50, is that it may prevent over tightening which can cause deformation to implant 100. As shown in FIG. 25, implant 100 has been expanded to a desired position and/or lordotic angle. Additionally, locking screw 50 has locked the relative position of the superior endplate 10 with respect to the inferior endplate 20 and the proximal portion of locking screw 50 has been broken off as explained above.

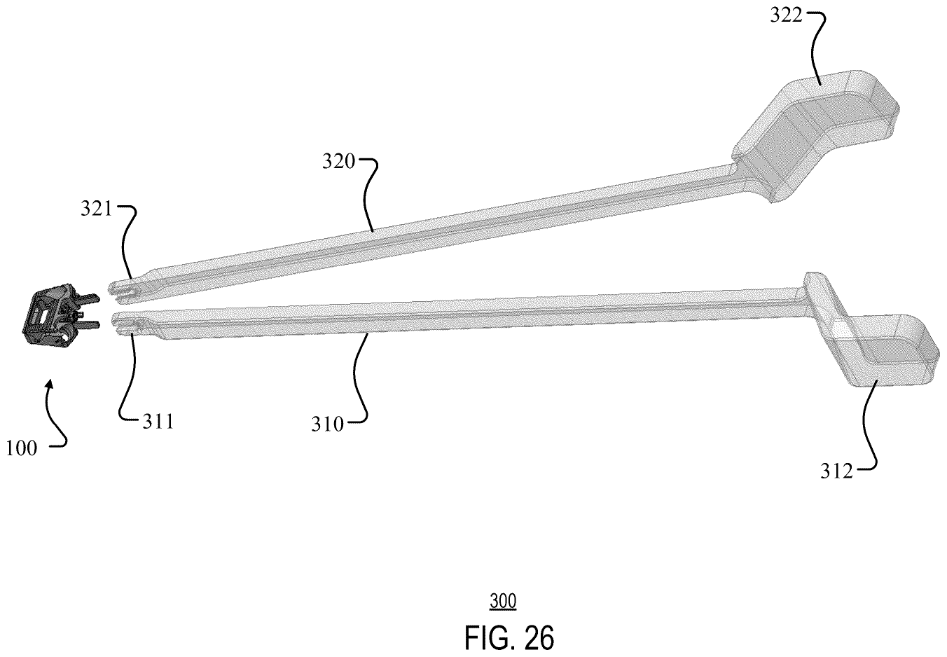

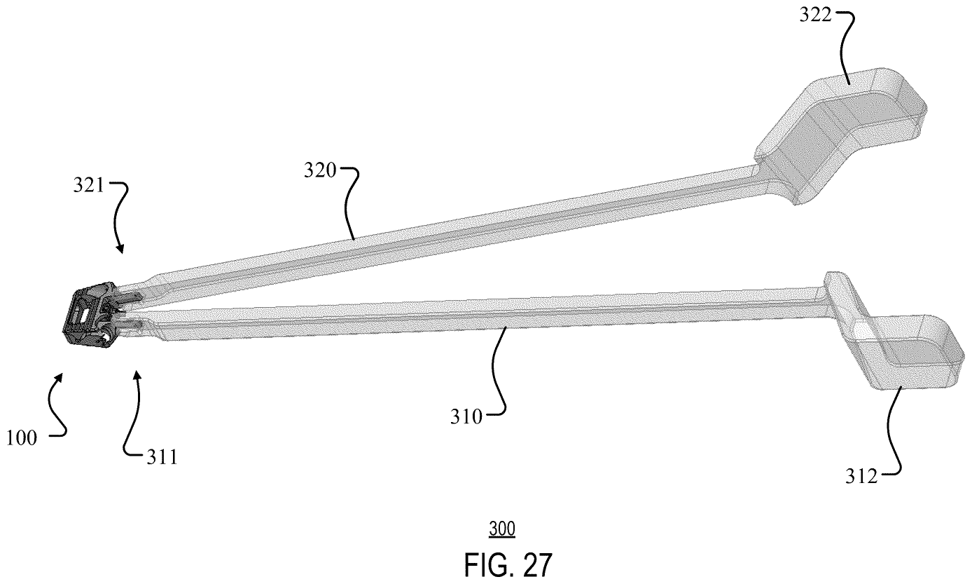

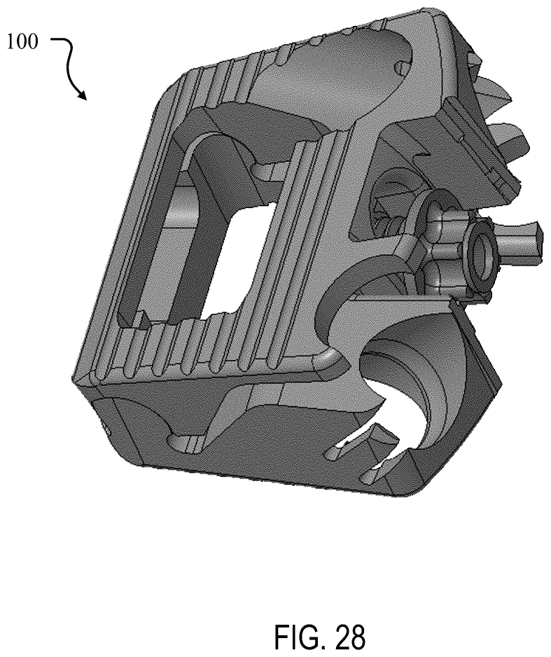

FIGS. 26 and 27 are various views of a surgical instrument 300 for use with disclosed expandable implants 100. In some embodiments, surgical instrument 300 may be referred to as a breakoff instrument and may be used to breakoff the mounting tangs 19, 29 of implant 100. In the example embodiment, surgical instrument 300 comprises a first instrument 310 and a second instrument 320. First instrument 310 may extend in a longitudinal direction from handle 312 towards gripping end 311. Similarly, second instrument 320 may extend in a longitudinal direction from handle 322 to gripping end 321. Gripping ends 311, 321 may comprise a channel having a size and shape generally corresponding to mounting tangs 19, 29. For example, as seen best in FIG. 27, the mounting tangs 19, 29 may be insert inside of the corresponding channels of gripping ends 311, 321. After the mounting tangs 19, 29 are nested within gripping ends 311, 321 an end user may push laterally outward and/or inward against handles 312, 322 to breakoff the corresponding mounting tang 19, 29. For example, as shown in FIG. 28 expandable implant 100 is in an expanded and lordosed configuration and the proximal portion of locking screw 50 and tangs 19, 29 have been broken off.

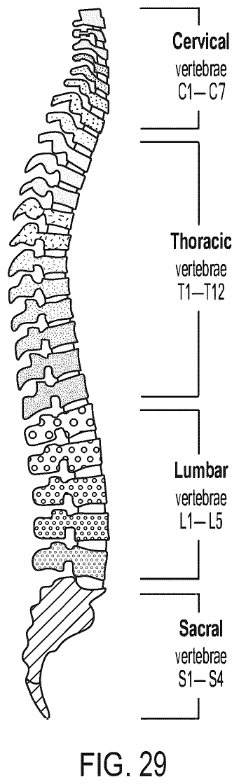

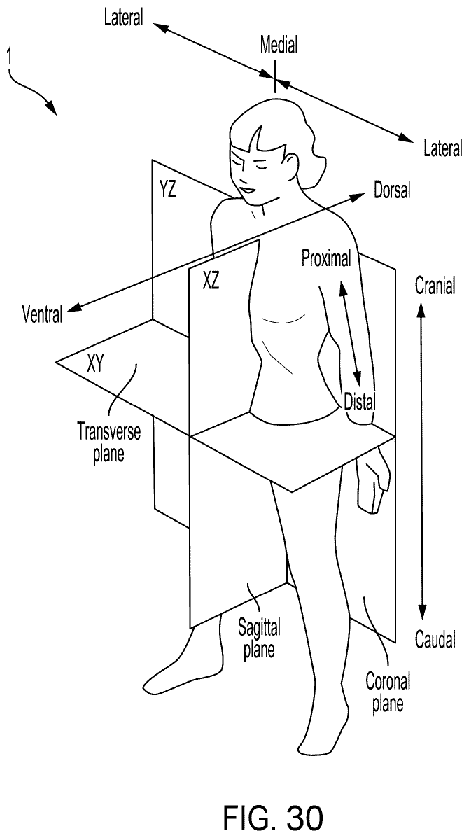

FIG. 29 is a reference drawing showing the human spine of which various disclosed implant embodiments may be installed in. FIG. 30 is a reference drawing showing various planes and reference directions of which the various disclosed implant embodiments may move in or act in with reference to a patient 1.

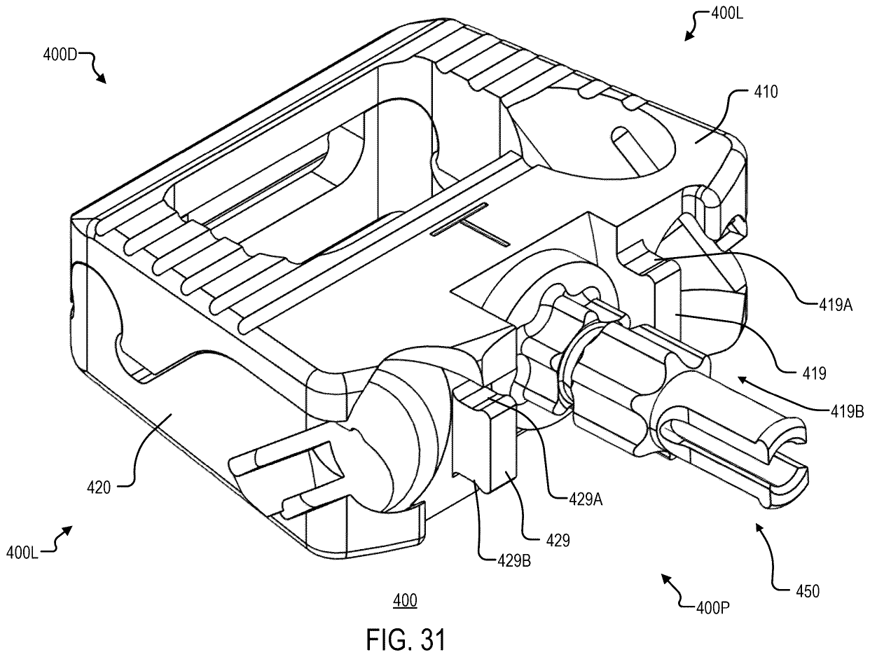

Referring generally to FIGS. 31-37 a second implant 400 embodiment is disclosed. Implant 400 may include the same, similar, and/or substantially the same components and functionality as explained above with respect to implant 100. Accordingly, duplicative description will be omitted. It shall be understood that various components and functionality of implant 100 are readily combinable with implant 400 and vice versa unless the context clearly indicates otherwise.

FIG. 31 is a perspective view of a second implant 400 embodiment. In this embodiment, implant 400 extends in a proximal-to-distal direction between a proximal end 400P and a distal end 400D and extends in a width-wise direction between a first lateral end 400L and a second lateral end 400L. Additionally, implant 400 includes a superior endplate 410 and an inferior endplate 420 having substantially similar features and functionality as explained above with respect to superior endplate 10 and inferior endplate 20 of implant 100, for example. However, in this embodiment, superior endplate 410 may include a first griping protrusion 419 extending in a proximal direction from the proximal end 400P of the superior endplate 410. Similarly, inferior endplate 420 may include a second griping protrusion 429 extending in a proximal direction from the proximal end 400P of the inferior endplate 420. In this embodiment, a size and shape of first gripping protrusion 419 is substantially the same as a size and shape of the second gripping protrusion 429. However, in other embodiments the first and second gripping protrusions 419, 429 may be differently sized and shaped, e.g., to bias the implant towards a surgical instrument and/or orientation for insertion. In this embodiment, and in the closed position, each gripping protrusion 419, 429 is disposed at approximately the same distance from an axis of rotation of breakoff set screw 450. In this embodiment, gripping protrusions 419, 429 may replace the need for the tangs 19, 29 of implant 100, for example. However, the concepts of utilizing breakoff tangs 19, 29 and gripping protrusions 419, 429 are not necessarily mutually exclusive and attributes of one may be combined and/or modified in view of the other.

In various embodiments, gripping protrusions 419, 429 may include various types of contouring to facilitate grasping of gripping protrusions 419, 429 with a corresponding inserter, for example surface indentations, surface outdents, channeling, etc. In the example embodiment, gripping protrusion 419 comprises a superior gripping surface 419A including an indented portion and an outdented chamfered portion at a proximal most end thereof, for example. Additionally, gripping protrusion 419 comprises an inferior gripping surface 419B including an indented portion and an outdented chamfered portion at a proximal most end thereof, for example. Likewise, gripping protrusion 429 comprises a superior gripping surface 429A including an indented portion and an outdented chamfered portion at a proximal most end thereof, for example. Additionally, gripping protrusion 429 comprises an inferior gripping surface 429B including an indented portion and an outdented chamfered portion at a proximal most end thereof, for example. In this way, gripping protrusions 419 and 429 are shaped like dovetails and a corresponding inserter tool may comprise a corresponding shaped dovetail groove which may grasp onto and/or slip over gripping protrusions 419 and 429 (not illustrated).

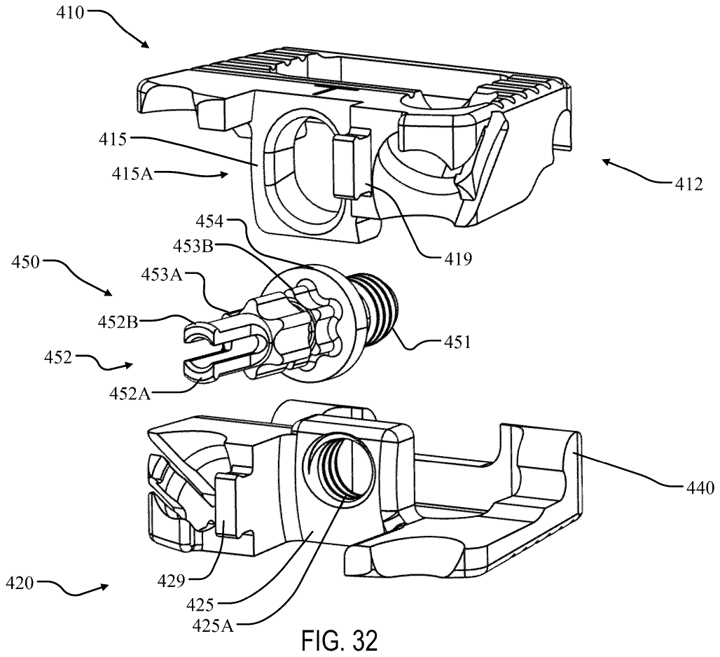

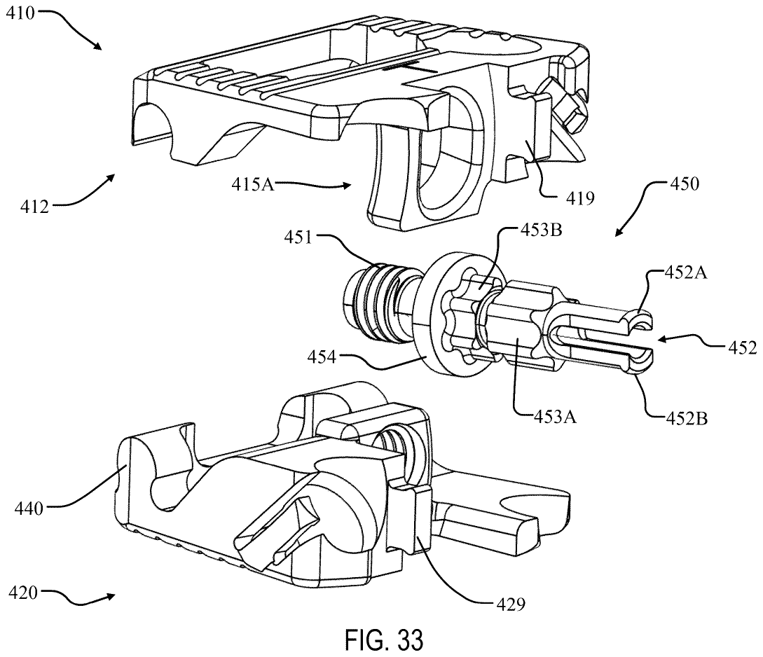

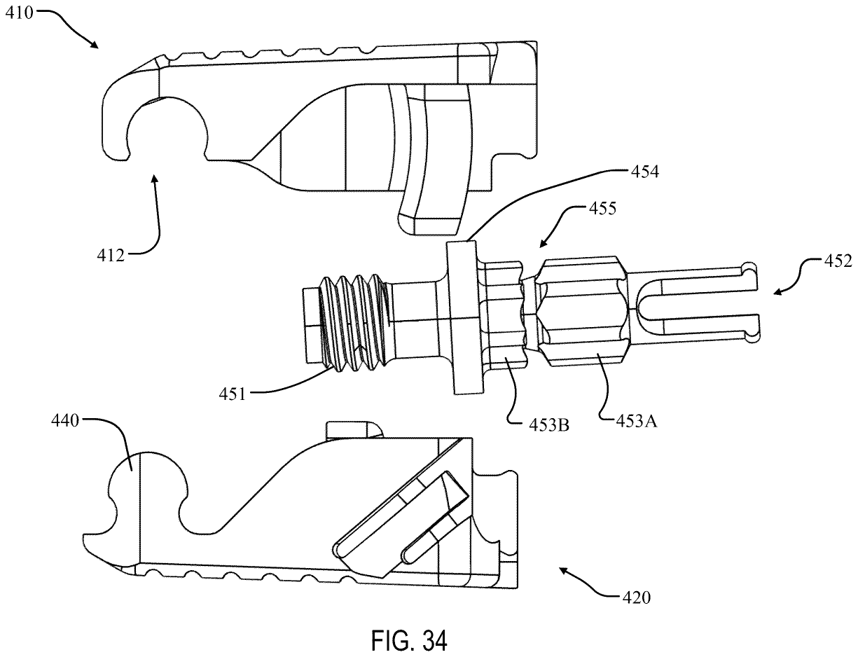

Referring generally to FIGS. 32, 33, and 34 various exploded parts views of implant 400 are illustrated. FIG. 32 is a first perspective exploded parts view of implant 400, FIG. 33 is a second perspective exploded parts view of implant 400, and FIG. 34 is a side exploded parts view of implant 400. In the example embodiment, the superior and inferior endplates 410, 420 of implant 400 may be hingedly coupled together by hinge member 440 and arcuate channel 412 having similar attributes as explained above with respect to hinge member 40 and channel 12 of implant 100, for example. Additionally, superior endplate 410 may also include a core 415 having an aperture 415A and inferior endplate 420 may include a core 425 having a threaded aperture 425A having similar attributes to core 15 and core 25 as explained above with respect to implant 100, for example. In the example embodiment, implant 400 utilizes a breakoff screw 450 for locking of a position of the superior and inferior endplate 410, 420. Breakoff screw 450 may include an external thread pattern 451 on an outside circumferential surface thereof, for example. The external thread pattern 451 of breakoff screw 450 may have a size and shape generally corresponding to the threaded aperture 425a of core 425 of the inferior endplate 420, for example. In various embodiments, an engagement surface 454 may be disposed adjacent and proximal of external thread pattern 451. In the example embodiment, engagement surface 454 is shaped like a washer and is directly connected to breakoff screw 450. However, in other embodiments, engagement surface 454 may be a washer or separated element, for example. In some embodiments, engagement surface 454 may be conically shaped. In the example embodiment, engagement surface 454 may include a relatively planar and/or flat distal surface and/or proximal surface.

In various embodiments, a proximal end of breakoff screw 450 may include a first flexible tang 452A and a second flexible tang 452B defining a discontinuous cylindrical shaped aperture 452 therebetween. Additionally, the first flexible tang 452A and second flexible tang 452B may each include an outdent at a proximal end thereof that is shaped like a segment of an annular ring. In the example embodiment, the first flexible tang 452A may flex inward towards the second flexible tang 452B under loading and vice versa due to the gap between them. At least one advantage of this configuration is that it may facilitate securing breakoff screw 450 to a corresponding drive tool (not illustrated) and the retention of the broken off part. For example, a drive tool may comprise a drive end having a female cavity with a corresponding size and shape to the drive features 453A, 453B. In various embodiments, the cavity may include a pair of indentations corresponding in size and shape to the outdents of flexible tangs 452A and 452B, for example. In use, an end user may align the flexible tangs 452A, 452B with the cavity, push down against the flexible tangs 452A, 452B which may cause them to flex inward towards each other such that they may slide within the cavity until the outdents of flexible tangs 45A and 452B are seated within the corresponding indents of the drive tool. Thereafter, an end user may continue to rotate and or tighten breakoff screw 450. In this way, after a proximal portion of breakoff screw 450 is broken off it may remain retained by the inserter due to the flexible tangs 452A and 452B being seated within the corresponding indents.

In some embodiments, aperture 452 may be understood as a cylindrical protrusion extending in a proximal direction and having a first slit and a second slit extending along the length thereof such that the cyldrical protrusion is compressible. In the example embodiment, a first drive feature 453A and a second drive feature 453B may be disposed adjacent to and distally with respect to a proximal most end of breakoff screw 450. Additionally, first and second drive features 453A, 453B may be disposed proximally with respect to engagement surface 454. In various embodiments, a breakoff location may be positioned between and/or adjacent to drive features 453A, 453B, which will be explained in further detail below. In the example embodiment, drive features 453A, 453B take a hexalobular shape, although various other shapes such as hexaganol, polygonal, torx, etc. are also contemplated. In some embodiments, a surgical drive tool having a corresponding socket may be coupled to drive features 453A and/or 453B to cause rotation of breakoff screw 450. Similarly as explained above with respect to locking screw 50, once breakoff screw 450 has been sufficiently tightened a proximal portion may breakoff and/or shear off while the distal portion may remain coupled to implant 400 locking a relative orientation of superior endplate 410 and inferior endplate 420 in place.

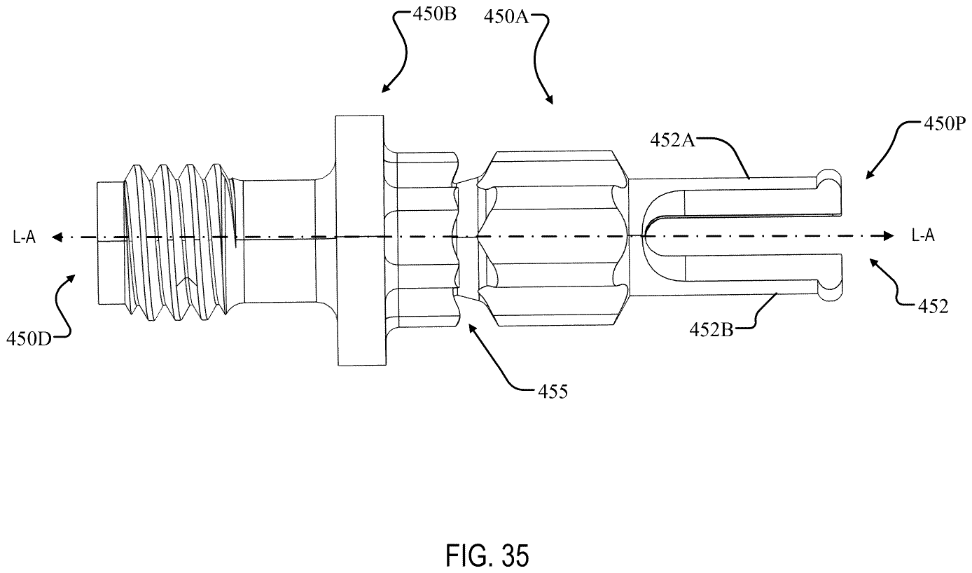

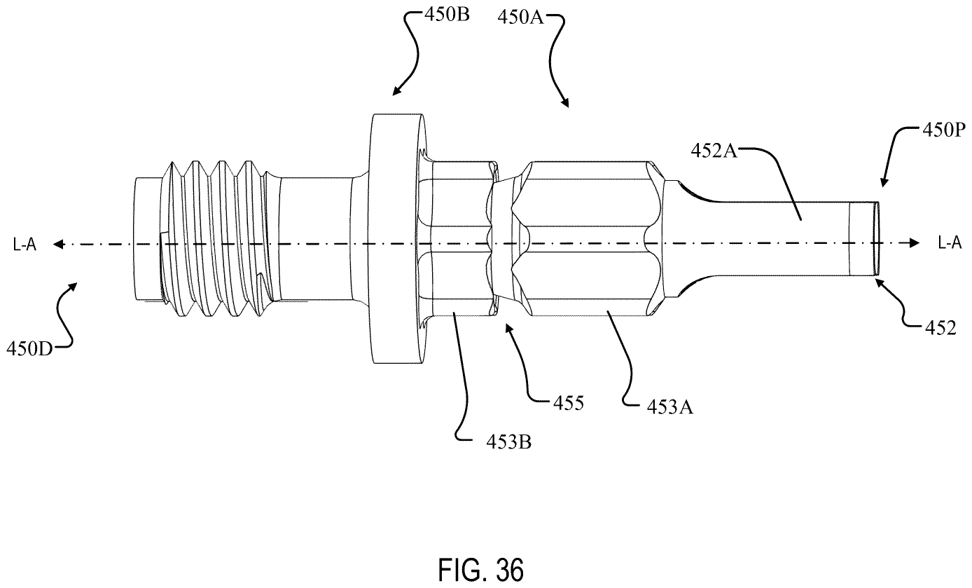

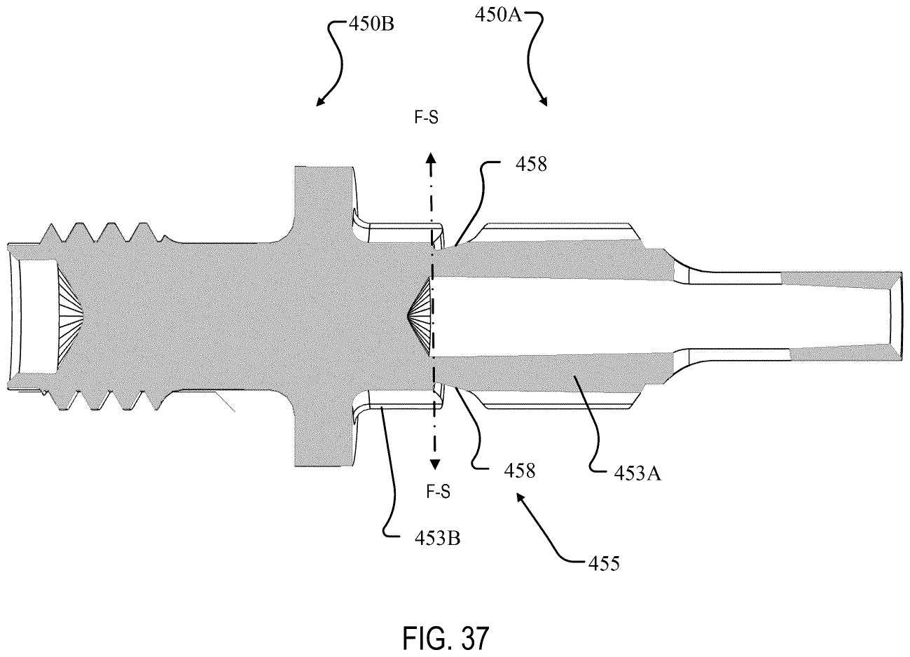

As seen best in FIGS. 35-37, breakoff screw 450 may extend in a longitudinal direction along a longitudinal axis L-A that is coaxially aligned with breakoff screw 450. FIG. 35 is a first side view of breakoff screw 450 in which the superior surface 452A and inferior surface 452B of discontinuous aperture 452 are visible. FIG. 36 is a second side view of a breakoff screw 450 that is rotated about 90 degrees with respect to FIG. 35 in which only the superior surface 452A is visible. With reference to FIG. 37, in various embodiments, breakoff location 455 may comprise a recessed fracture surface F-S that is inset with respect to a leading edge (proximal most edge) of drive feature 453A. At least one advantage of the recessed fracture surface may be that delicate tissue is prevented and/or suppressed from coming into contact with relatively sharp ends of the fracture surface. In the example illustration, a relative location of the recessed fracture surface is represented by dashed lines F-S. In the example embodiment, breakoff location 455 may be considered as the boundary between a proximal portion 450A and a distal portion 450B of breakoff screw 450, for example. In this embodiment, the boundary between drive features 453A and 453B comprises a necked down portion 458 extending from a distal end of drive feature 453A to an inset portion of drive feature 453B that is inset with respect to an outermost and/or proximal most surface of drive feature 453B thereby defining a portion of breakoff set screw 450 having a minimum cross sectional diameter. Accordingly, when breakoff screw 450 is sufficiently tightened within threaded aperture 425A of core 425 such that the breakoff location 455 experiences a sufficient torque the proximal portion 450A may breakoff from the distal portion 450B. For example, when a sufficient rotational force is applied to the proximal end of breakoff screw 450 while a distal end of breakoff screw 450 is stationary, i.e., when breakoff screw 450 is secured in a locked position and a continued rotational force (torque) is applied to the proximal end of breakoff screw 450 the drive feature 453A and cylindrical end having the discontinuous aperture 452 may breakoff. For further explanation in the similar context of implant 100, see FIGS. 10 and 11 and the corresponding discussion thereof.

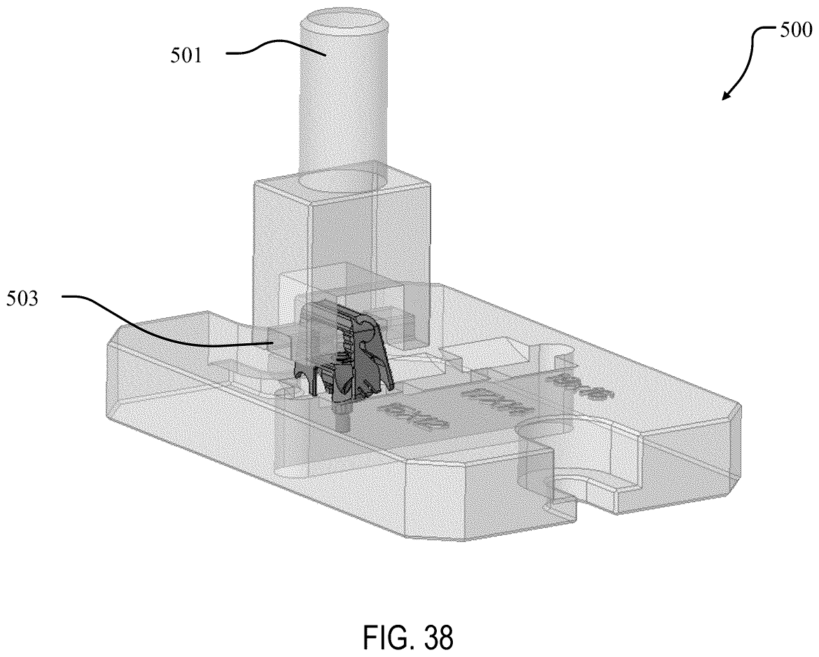

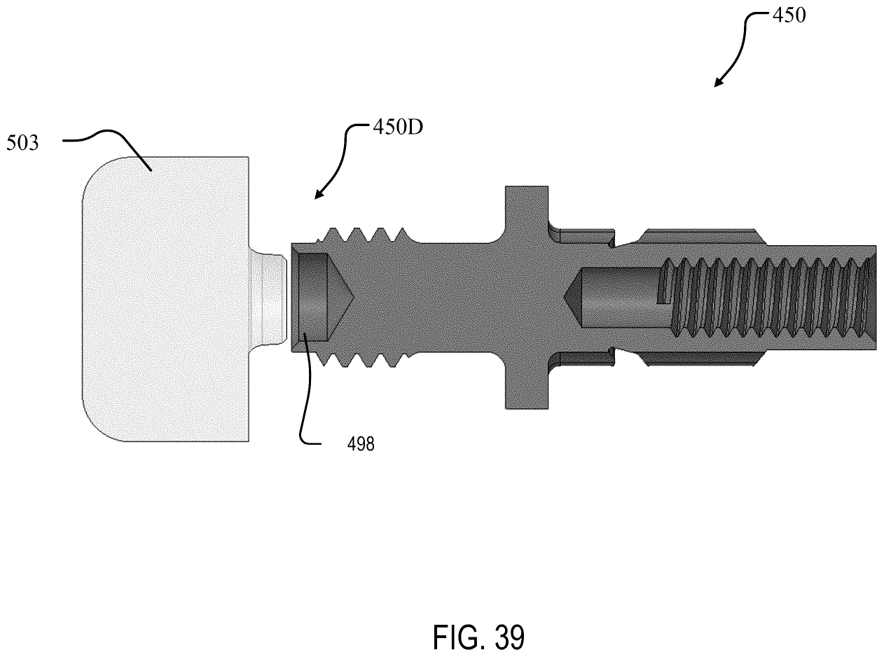

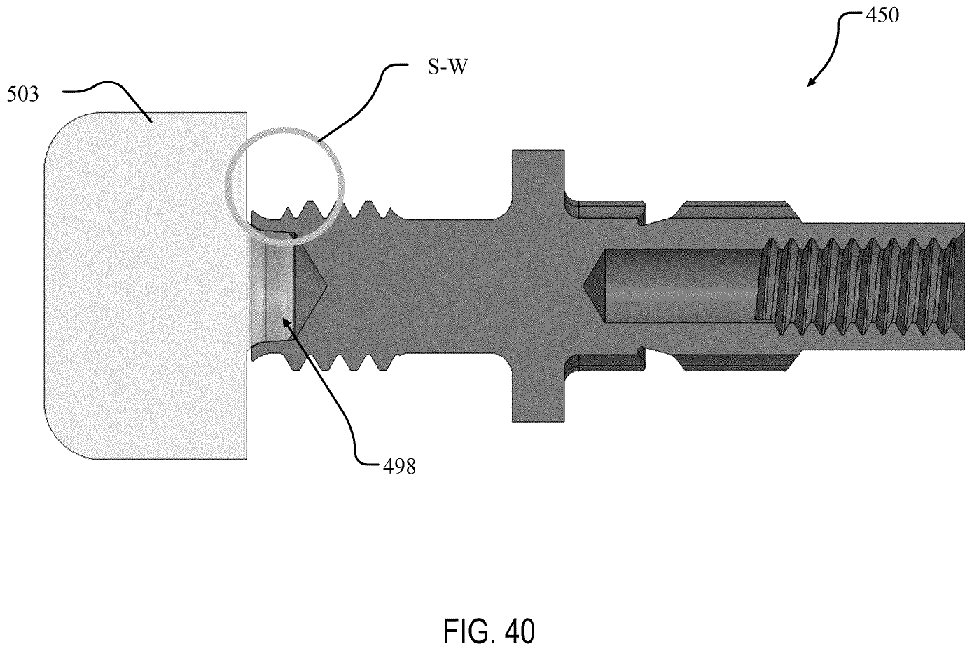

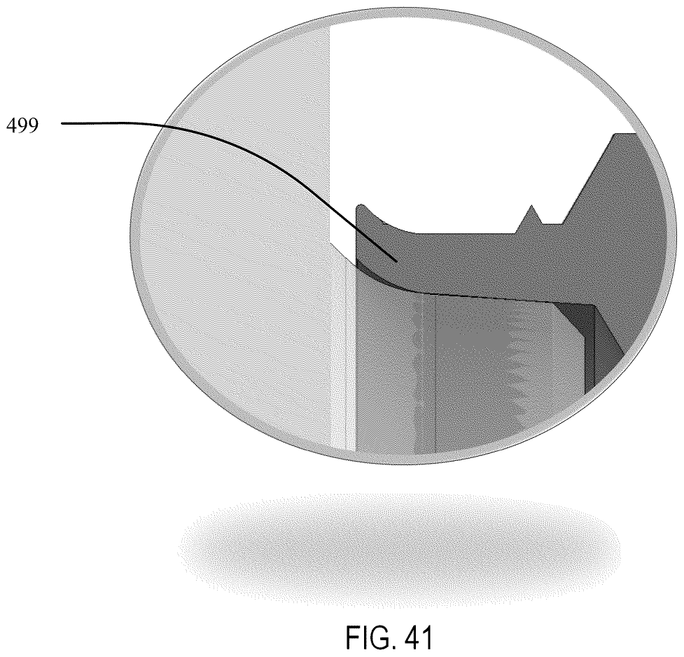

Referring to FIGS. 38-42 an example swaging process is performed to a distal most end of breakoff screw 450. FIG. 38 is a perspective view of a swaging fixture 500, and FIG. 39 is a cross section view of a swage mandrel and a distal end 450D of a breakoff screw 450 before the commencement of a swage process. FIG. 40 is a cross section view showing a result of a swage process, and FIG. 41 is an enlarged view of region S-W of FIG. 40. In the example embodiment, swaging fixture 500 comprises a swaging ram 501 and a swaging mandrel 503 that are supported by the base of the apparatus. The swaging mandrel 503 may include an outdent that corresponds to and is slightly larger than the distal most indent 498 (swage bore) of breakoff screw 450, for example. As seen in region S-W of FIG. 40, when swaging mandrel 503 is advanced into the distal most indent 498 (swage bore) a flared out portion 499 (swaged portion) is formed at a distal most end of breakoff screw 450. An example advantage of a swaged end may be that it facilitates retention of the breakoff screw 450 such that it serves as a stopping structure preventing breakoff screw 450 from backing out of implant 100.

It shall be understood that although breakoff screw 450 is explained concurrently with implant 400 and in the context of an intervertebral implant, the concepts of breakoff screw 450 may be applied to other embodiments used for alternate purposes, for example for use in a pedicle screw to insert in a tulip to tighten down a rod. It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

Referring generally to FIGS. 42-51, a third implant 400Z and a surgical tool 600 for use with various implants is disclosed. FIGS. 42A-42B illustrate a third embodiment of an implant 400Z. Implant 400Z may have the same, similar, and/or substantially the same components and functionality as explained above with respect to implant 400, for example. Accordingly, duplicative description will be omitted. A difference may be that breakoff screw 450 does not include the discontinuous cylindrical shaped aperture 452 (see FIG. 34) having tangs 452A, 452B, for example. Rather, breakoff screw 450, to the extent included, may include a continuous cylindrical shaped aperture 452Z without the discontinuity. In some embodiments, a similar screw may be used that is not a breakoff screw but rather may be referred to as a locking screw. Additionally, in this embodiment, implant 400Z may include a first gripping indentation 419Z and a second gripping indentation 429Z (see FIG. 42A) in lieu of the gripping protrusions 419, 429 (as shown in FIG. 33), for example. In various embodiments, first gripping indentation 419Z may be formed as a part of the superior endplate 410 and second gripping indentation 429Z may be formed as a part of the inferior endplate 420, for example.

In various embodiments, gripping indentations 419Z, 429Z may include various types of contouring to facilitate coupling with a corresponding surgical tool 600. In this embodiment, gripping indentations 419Z, 429Z each comprise a slotted indentation extending in a proximal to distal direction with a curved superior surface 419S, 429S and a curved inferior surface 419I, 429I. Additionally, gripping indentations 419Z, 429Z each have an open channel portion 419X, 429X adjacent the breakoff screw 450, to the extent a breakoff screw is included. As will be explained in further detail below, corresponding gripping protrusions of a surgical tool 600 may have a substantially similar size and shape to the gripping indentations 419Z, 429Z, for example.

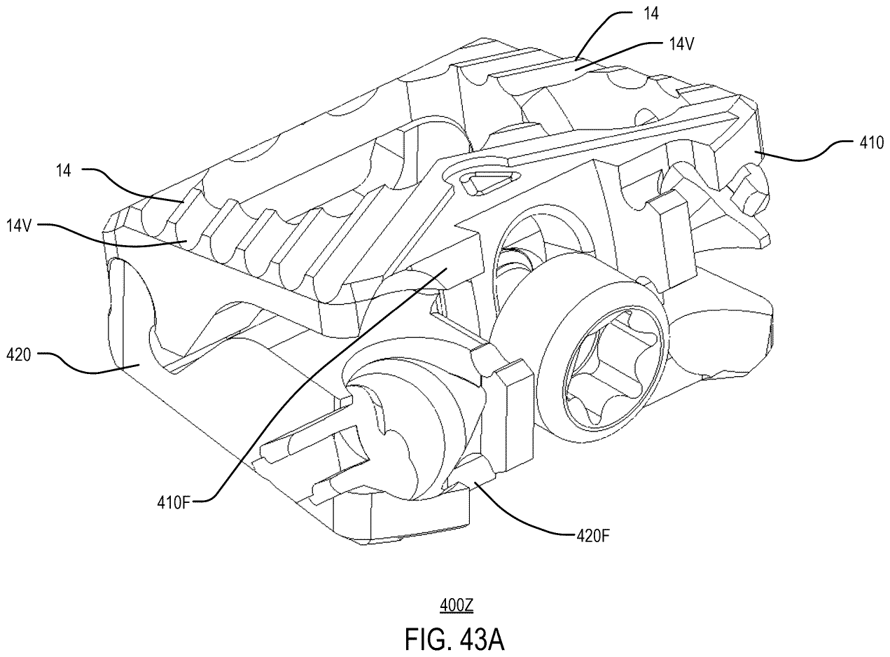

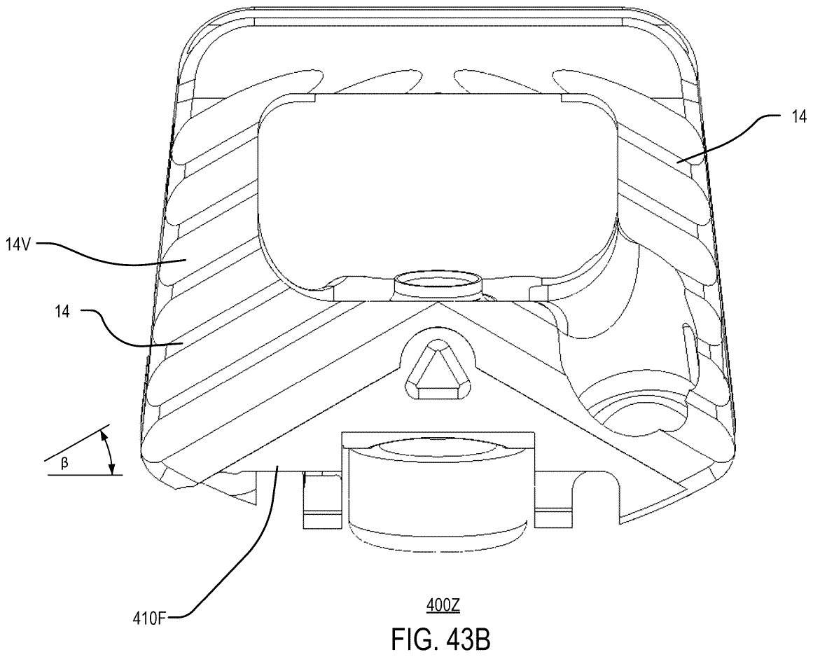

FIGS. 43A-43B illustrate a similar embodiment as FIGS. 42A-42B. As shown, the implant 400Z includes angled engagement features 14. In the example embodiment, engagement features 14 extend diagonally across the exposed uppermost surface of superior endplate 410 and across the exposed lowermost surface of inferior endplate 420. The engagement features 14 comprise flattened top rails that are sequentially spaced apart with rounded bottom valleys 14V therebetween. As seen best in the top-down view of FIG. 43B, the engagement features 14 of the superior endplate 410 are oriented at an angle β with respect to a proximal face 410F of the superior endplate 410. Similarly, the engagement features 14 of the inferior endplate 420 are oriented at an angle β with respect to a proximal face 420F of the inferior endplate 420 (see FIG. 43A). In various embodiments, the angle β may be about 20-40 degrees and in at least one embodiment the angle β may be angled at about 30 degrees. At least one advantage of this orientation may be a relatively greater resistance and/or suppression of expulsion of the implant 400Z relative to embodiments in which engagement features solely extend horizontally across the implant 400Z. In the example embodiment of FIGS. 43A-43B, by orienting the engagement features 14 diagonally, the implant 400Z may resist expulsion in multiple directions, e.g., forward flexion/extension and lateral bending.

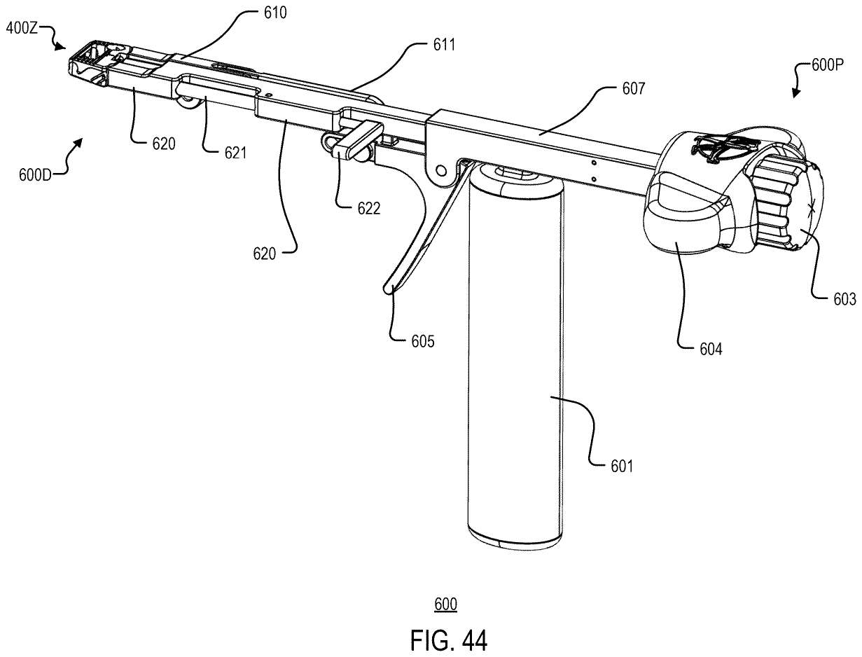

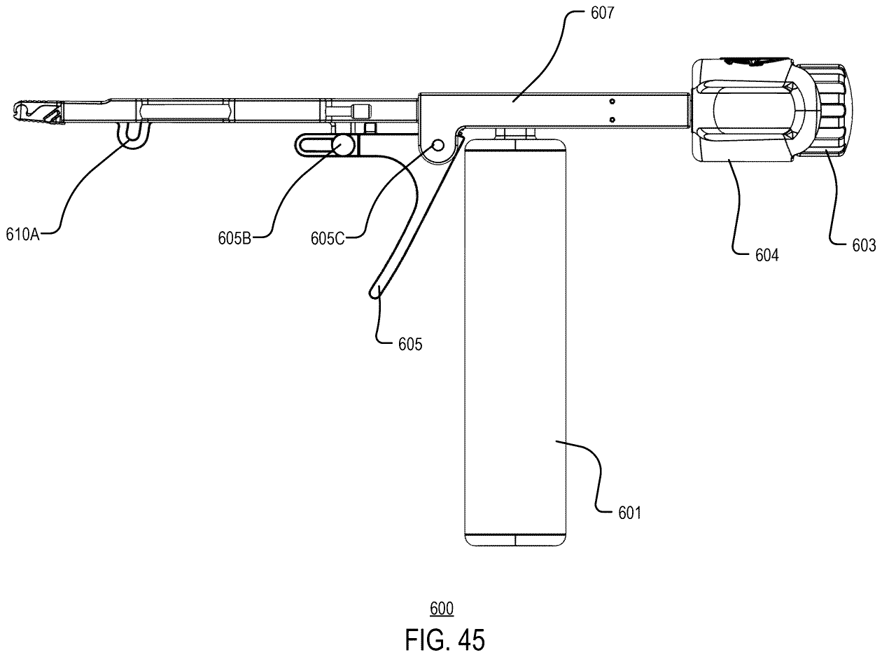

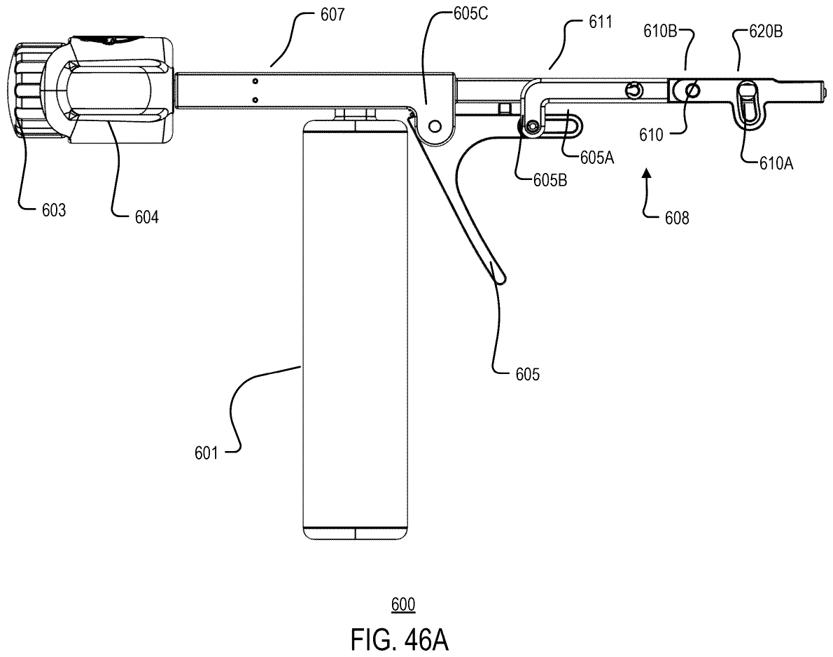

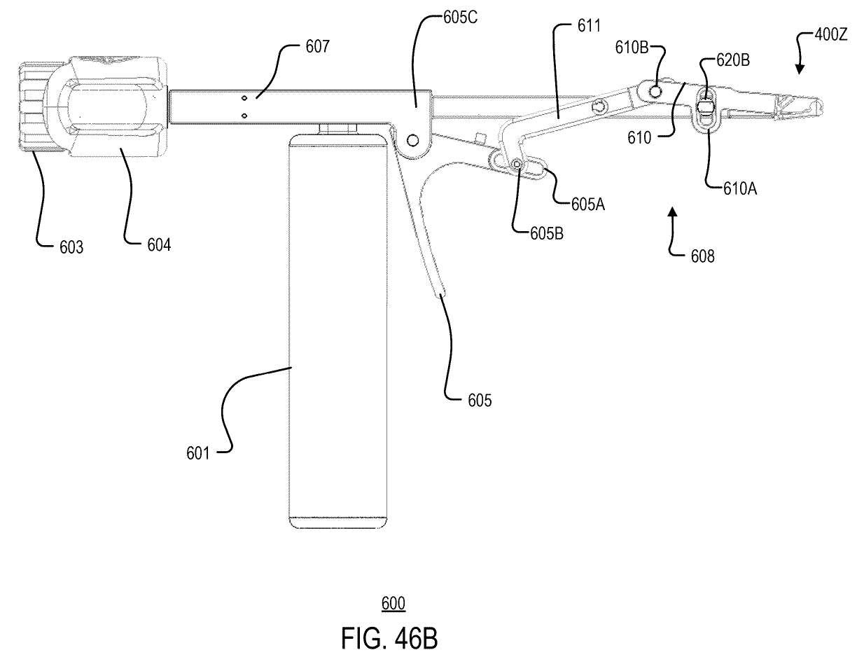

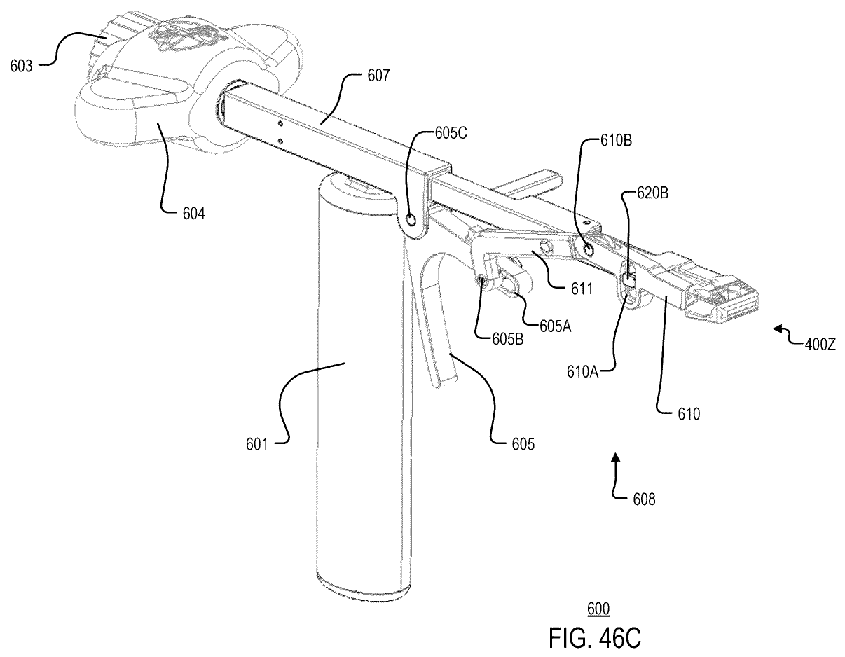

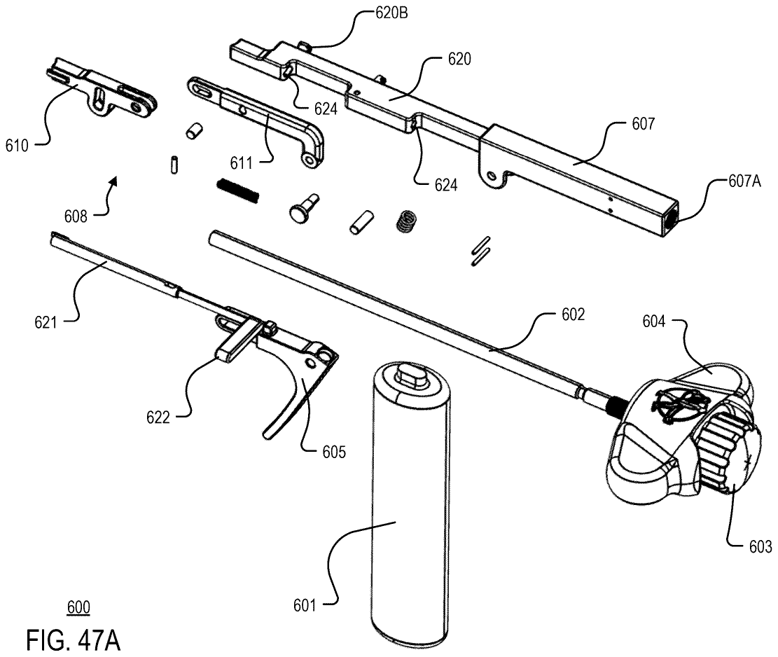

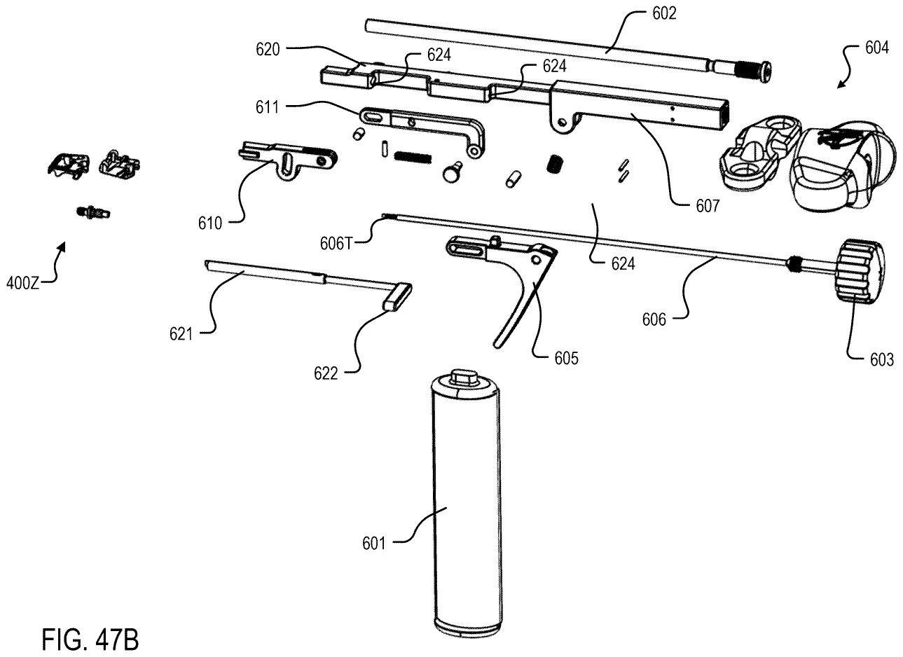

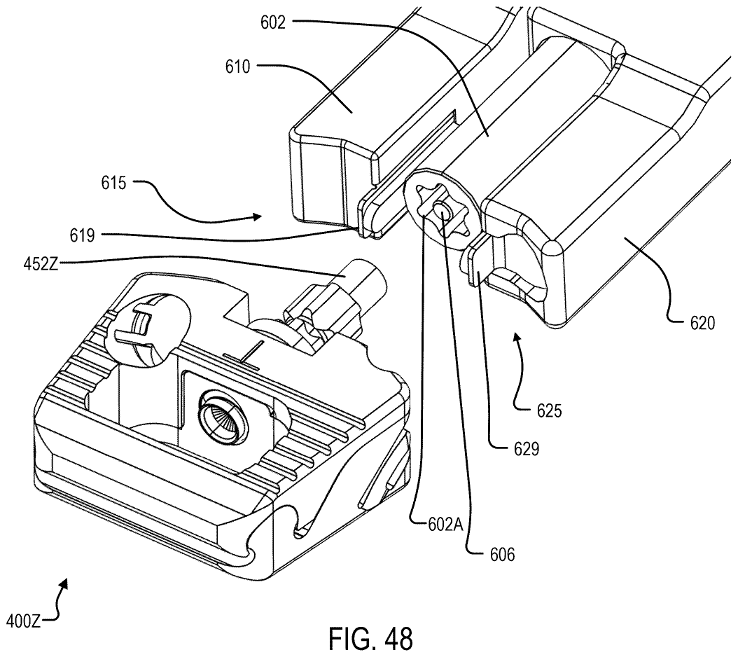

Referring generally to FIGS. 44-48, various views of a surgical tool 600 are disclosed. FIG. 44 illustrates the surgical tool 600 coupled to implant 400Z. FIG. 44 is a perspective view of surgical tool 600; FIG. 45 is a first side view of surgical tool 600; and FIG. 46A is a second side view of surgical tool 600. FIG. 46B is a third side view of surgical tool 600 in an operative position and FIG. 46C is a perspective view of surgical tool 600 in the operative position of FIG. 46B. FIG. 47A is an exploded parts view of surgical tool 600 with select parts removed for ease of understanding and FIG. 47B is an exploded parts view of surgical tool 600 showing additional detail and parts. FIG. 48 illustrates surgical tool 600 immediately prior to coupling with implant 400Z. In the example embodiment, tool 600 may serve several purposes. For example, tool 600 may be used to insert implant 400Z, expand implant 400Z, and in some embodiments may also rotate breakoff screw 450 to cause it to separate into two portions along breakoff location 455 (see FIG. 34).

With reference to the perspective view of FIG. 44 and the exploded parts views of FIGS. 47A and 47B, tool 600 may include a gripping handle 601 that is securely coupled to first body portion 607. First body portion 607 may rotatably support an inner shaft 606 and an outer shaft 602 therein. Shafts 606, 602 may extend through aperture 607A of first body portion 607 in a longitudinal direction from a proximal end to a distal end. The inner shaft 606 may be securely connected to turn knob 603 at a proximal end 600P and the outer shaft 602 may be securely connected to turn handle 604 at the proximal end 600P. The inner shaft 606 may extend through outer shaft 602 and include a thread pattern 606T at a distal end thereof for securely connecting to corresponding threads 456 of breakoff screw 450 (see FIG. 42B for example). The inner shaft 606 may be independently rotatable relative to outer shaft 602 by rotating turn knob 603. Additionally, the outer shaft 602 may be independently rotatable relative to inner shaft 606 by rotating turn handle 604, for example. Generally, both the inner shaft 606 and outer shaft 602 will interact with and/or couple to breakoff screw 450 as will be explained in further detail below.

With reference to FIGS. 45 and 46A-46C, tool 600 may include an actuator 605 in the form of a trigger that is connected to first body portion 607 at pin 605C. Actuator 605 may be actionable to expand implant 400Z by pivoting a linkage assembly 608 including first arm 610 and second arm 611 relative to a third fixed arm 620, for example. Second arm 611 may be coupled to actuator 605 via pin 605B such that pin 605B may be linearly translatable forward and backward in a proximal-to-distal direction within slotted aperture 605A. In turn, second arm 611 may be coupled to first arm 610 via pin 610B such that second arm 611 may pivot up and down. Additionally, first arm 610 may be coupled to third arm 620 and in various embodiments third arm 620 may be fixed relative to first body portion 607. Additionally, third arm 620 may include a protrusion 620B which may be slidably seated within a curved vertical slot, for example slot 610A, and thereby guide motion of arm 610 as it pivots up and down. As seen best in FIGS. 46B and 46C, depressing actuator 605 may cause linkage assembly 608 to pivot by causing second arm 611 to move distally such that first arm 610 pivots relative to third arm 620 and implant 400Z may be expanded.

With reference back to FIG. 44, third arm 620 may include an aperture extending therethrough in a proximal to distal direction for accommodating a linearly translatable shaft 621. Shaft 621 may independently move forward and backward in a proximal to distal direction within the through aperture 624 of third fixed arm 620 (see FIGS. 47A and 47B), for example. Shaft 621 may include a charging handle at a proximal end thereof allowing an end user to manipulate shaft 621 in a forward and backward motion (proximal motion and distal motion in the proximal-to-distal direction). In some embodiments, an end user may translate charging handle 622 rotatably in a clockwise or counterclockwise motion, for example to lock shaft 621 in a relative position. Additionally, in some embodiments, when shaft 621 is not in a locked position, depressing actuator 605 will not expand implant 400Z for reasons explained in more detail below.

With reference to FIG. 48, first arm 610 may include a gripping outdent 619 having a size and shape generally corresponding to a size and shape of gripping indentation 419Z. Additionally, first arm 610 may include a counter torque surface 615 having a size and shape generally corresponding to a size and shape of the proximal surface of the superior endplate 410, for example. Similarly, shaft 621, extending through fixed arm 620, may include a gripping outdent 629 having a size and shape generally corresponding to a size and shape of gripping indentation 429Z (as shown in FIG. 42A). Additionally, second arm 620 may include a counter torque surface 625 having a size and shape generally corresponding to a size and shape of the proximal surface of the inferior endplate 420, for example. In this way, when shaft 621 is moved distally such that the outdent 629 is seated within indent 429Z and outdent 619 is seated within indentation 419Z (as shown in FIG. 42A) an end user may activate actuator 605 to expand implant 400Z while surfaces 615, 625 resist a twisting motion of implant 100. For example, as shown in FIGS. 46B and 46C an end user may depress actuator 605 by squeezing the trigger which in turn may cause the linkage assembly 608 to pivot the first arm 610 relative to fixed arm 620 such that tool 600 may urge the superior endplate 410 and inferior endplate 420 away from one another.

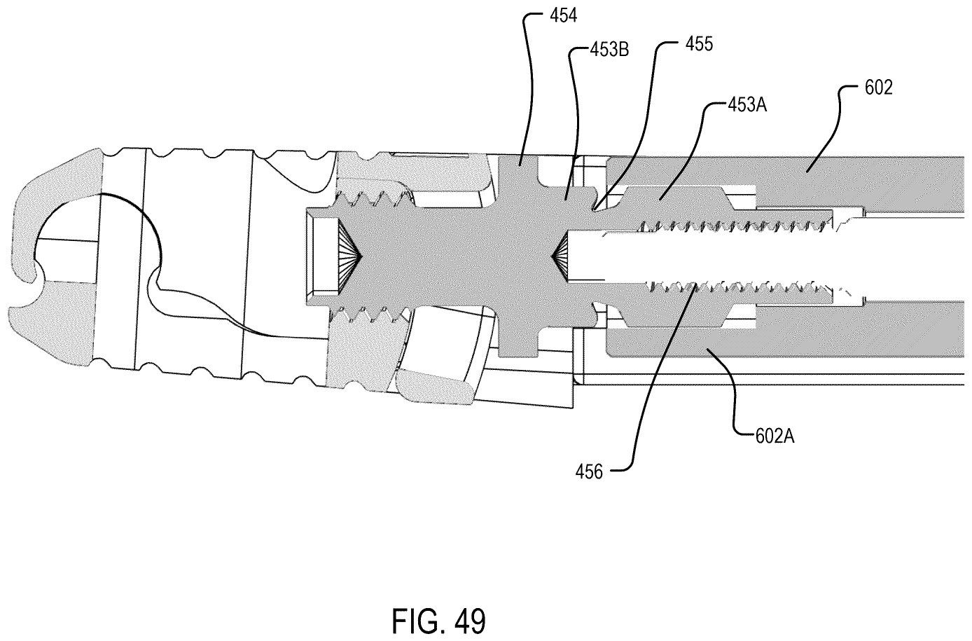

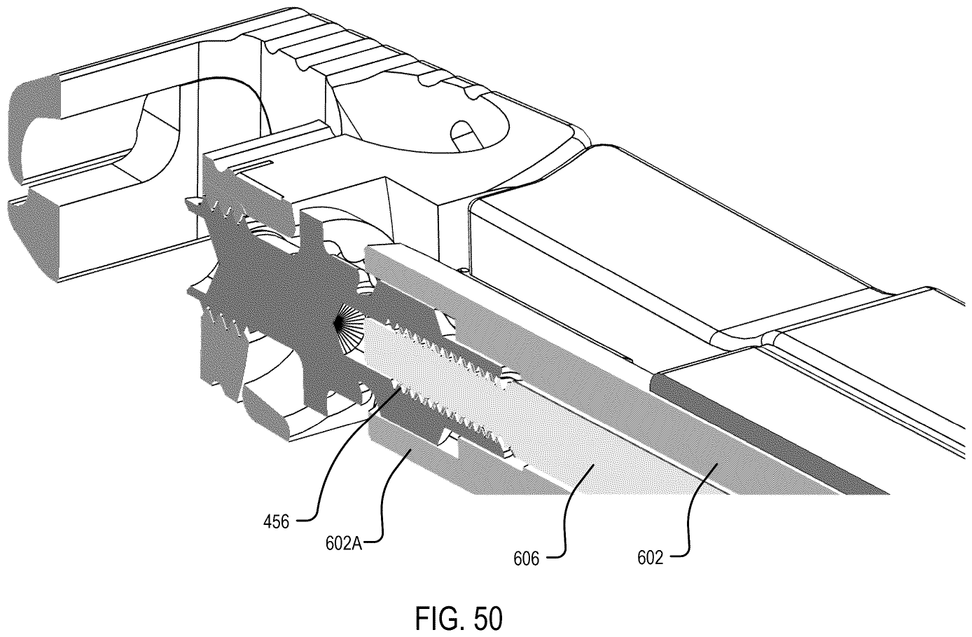

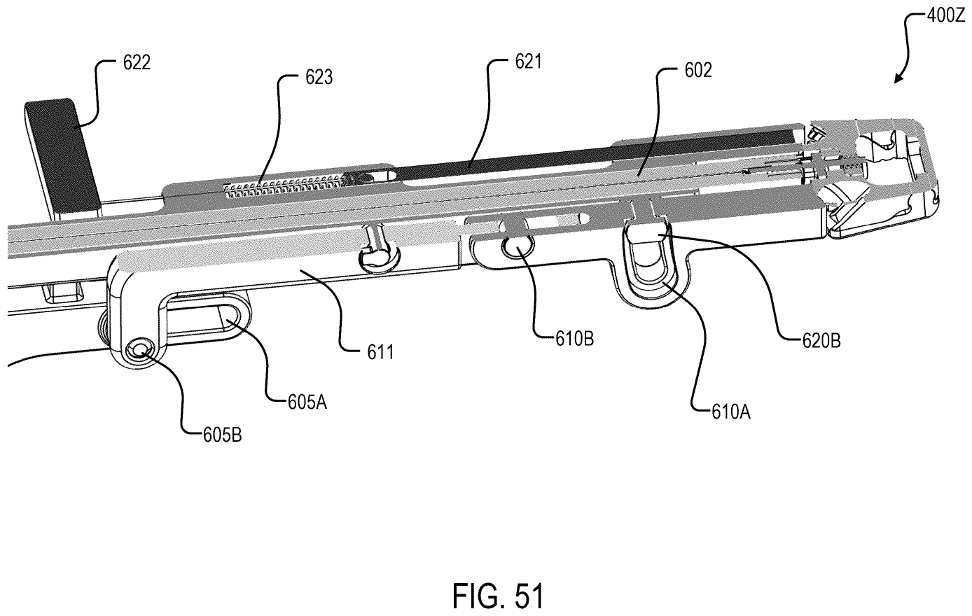

With reference to the cross-section drawings of FIGS. 49, 50, and 51 additional features and functionality of tool 600 will be explained. In FIGS. 49 and 50, it is shown that the threaded end of inner shaft 606 is threadably engaged with the threaded aperture 456 of breakoff screw 450. Additionally, a proximal drive feature 453A is mated within a corresponding female drive feature 602A (see FIG. 48) at the distal most end of outer shaft 602. As explained previously, inner shaft 606 and outer shaft 602 are independently operable. In use, an end user may initially position inner shaft 606 adjacent to/within the front portion of threaded aperture 452 and rotate inner shaft 606 via turn knob 603. As inner shaft 606 is rotated, implant 400Z is pulled towards tool 600. FIGS. 49-51 show implant 400 after inner shaft 606 has been sufficiently rotated such that implant 400Z abuts the counter torque surfaces 615, 625 and the gripping protrusion 619 may be seated within the corresponding gripping indentation 419Z. With reference to FIG. 51, once implant 400Z is pulled sufficiently towards tool 600, an end user may actuate shaft 621 by pushing on charging handle 622 to overcome the biasing force of spring 623 such that gripping protrusion 629 may be seated within the corresponding gripping indentation 429Z, for example. In some embodiments, an end user may rotate charging handle 622 such that biasing force of spring 623 is prevented from urging shaft 621 away from implant 400Z. At this point, the implant 400Z may be in an operatively engaged position with tool 600.

Once implant 400Z is in the operatively engaged position, an end user may insert implant 400Z into a disc space between a superior vertebra and inferior vertebra (cephalad vertebra and caudal vertebra), for example. After the implant 400Z is positioned between the superior and inferior vertebrae, an end user may depress actuator 605 thereby causing the linkage assembly 608 to pivot and spread the superior endplate 410 apart from the inferior endplate 420. Once the end user has expanded implant 400Z to an appropriate position, the end user may rotate turn handle 604 which will rotate breakoff screw 450 via first drive feature 453A. The end user may continue to rotate breakoff screw 450 such that it advances into implant 400Z in a proximal to distal direction and locks implant 400Z in the expanded configuration similarly as explained above. The end user may continue to rotate turn handle 604 until the torque applied to breakoff screw 450 is great enough that the breakoff screw 450 will shear into two pieces similarly as explained above. Notably, the broken off or sheared off portion of breakoff screw 450 may be retained by tool 600 on account of inner shaft 606 being threadably coupled to threaded aperture 452.

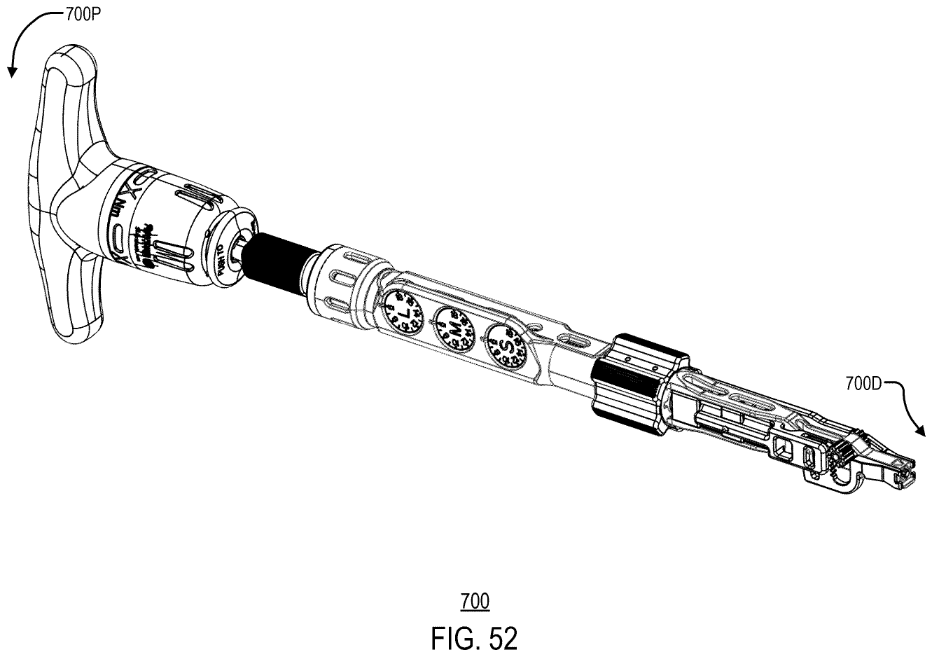

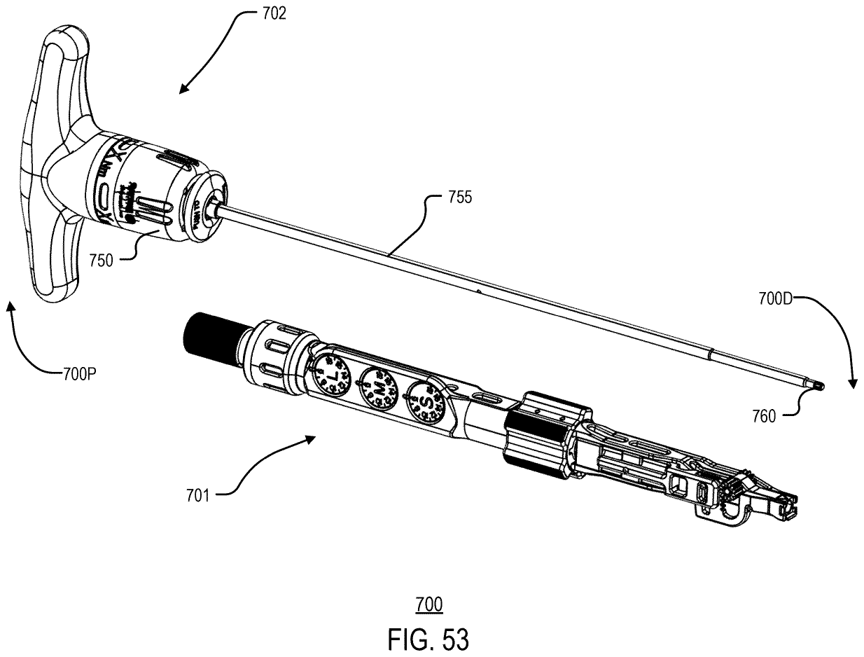

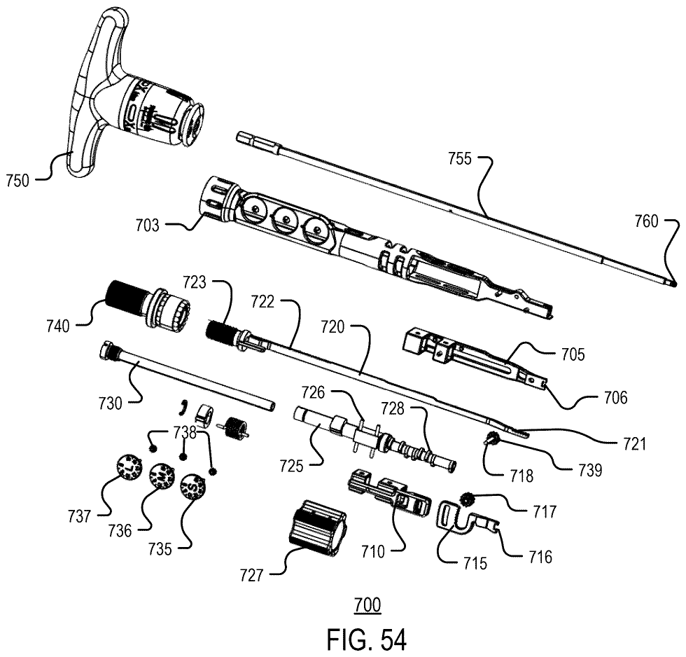

With reference to the perspective views of FIGS. 52 and 53 and the exploded parts view of FIG. 54, a surgical tool 700 is disclosed. Surgical tool 700 may be used to insert any of the various implant embodiments disclosed herein into a disc space and expand the implant. Additionally, surgical tool 700 may be used to lock various implant embodiments in an angled configuration after the implant has been expanded. Surgical tool 700 may include an inserter portion 701 having an outer body assembly 703 for gripping, inserting, and expanding an implant and a drive portion configured for disposal within the inserter portion 701 for locking the implant in an expanded position. Drive portion 702 may be disposed within the hollow shaft of the inserter portion and may include a drive actuator 750 and a drive shaft 755 having a drive end 760. Inserter portion 701 may include a rotation knob handle 740 that is threadably engaged with or operatively coupled with a gear rack 720 having a threaded tube on the proximal gear rack end 723. In the example embodiment, gear rack tube 730 is operatively coupled with cam shaft 725 and is coaxially positioned within a tube of the proximal gear rack end 723.

Cam shaft 725 may include a plurality of cam shaft lobes 728 protruding transverse from a longitudinal direction of the shaft 725 that may be disposed in the distal portion of cam shaft 725. In various embodiments, the number of cam shaft lobes 728 may be about 4-6 and in others there may be about 2 to about 10. Those with skill in the art will recognize that the number of cam shaft lobes 728 may vary. In some embodiments, the cam shaft lobes 728 and the cam shaft 725 may be formed by a unitary piece, e.g., they may be monolithically formed. In other embodiments, the cam shaft lobes 728 may be modularly integrated as needed on the distal portion of cam shaft 725. In various embodiments, the cam shaft lobes 728 may be protruding at about 180° offsets from each other in the lateral directions.

In the example embodiment, non-rotating jaw 705 may be secured and in contact with a lateral side of the distal end of gear rack 720 and the cam shaft lobes 728. Rotating jaw slide 710 and rotating jaw 715 assembly are secured and in contact with opposite lateral sides of the cam shaft lobes 728. In various embodiments, drive shaft 755 includes a drive end 760 that may coaxially extend through a respective central aperture of rotation knob handle 740, proximal gear rack end 723, gear rack tube 730, and cam shaft 725 in a longitudinal direction from a proximal end to a distal end. The drive shaft 755 may be operatively connected to a drive actuator 750 at the proximal end 700P for rotating the drive shaft 755. In various embodiments drive actuator 750 may be a T-shaped handle. Drive shaft 755 may include a drive end 760 disposed at a distal end of the surgical tool 700 for securely connect to a corresponding locking screw drive feature 453B. In various embodiments, the drive shaft 755 may include a drive end 760 having a size and shape corresponding to a size and shape of drive feature 453B (see FIG. 32). In various embodiments, the inner drive shaft 755 may be independently rotatable relative to outer gear rack tube 730 and cam shaft 725 by activating the drive actuator 750. Additionally, the cam shaft 725 may be rotatable relative to inner drive shaft 755 by cam twist grip 727 which may be securely coupled to cam shaft 725 by cam shaft pins 726. Generally, both the inner drive shaft 755 and outer cam shaft 725 will interact with and/or couple to implant 400Z as will be explained in further detail below.

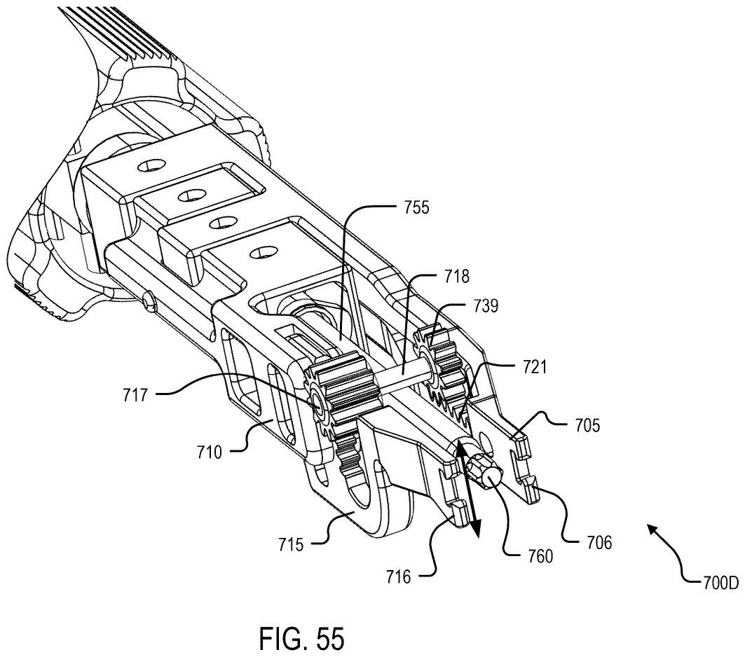

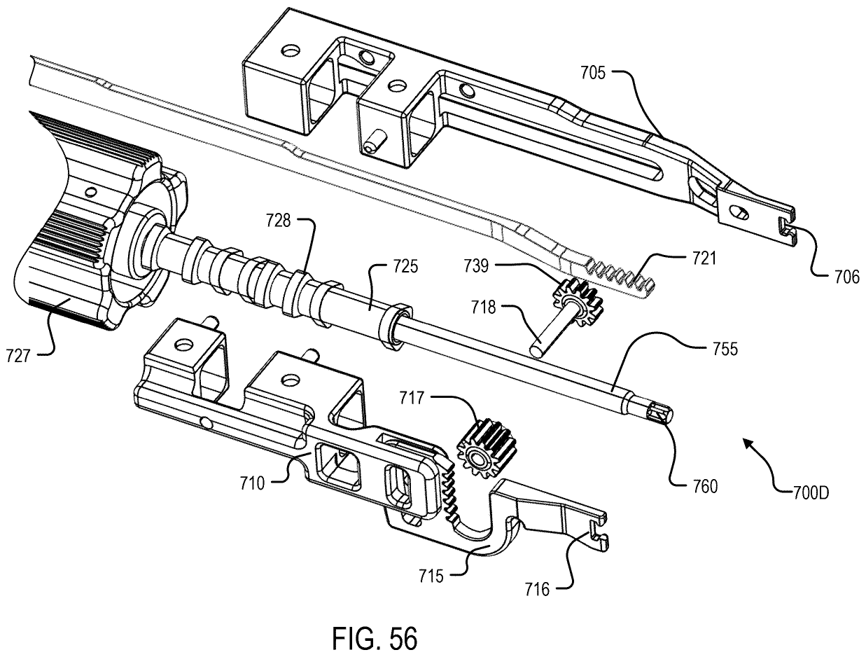

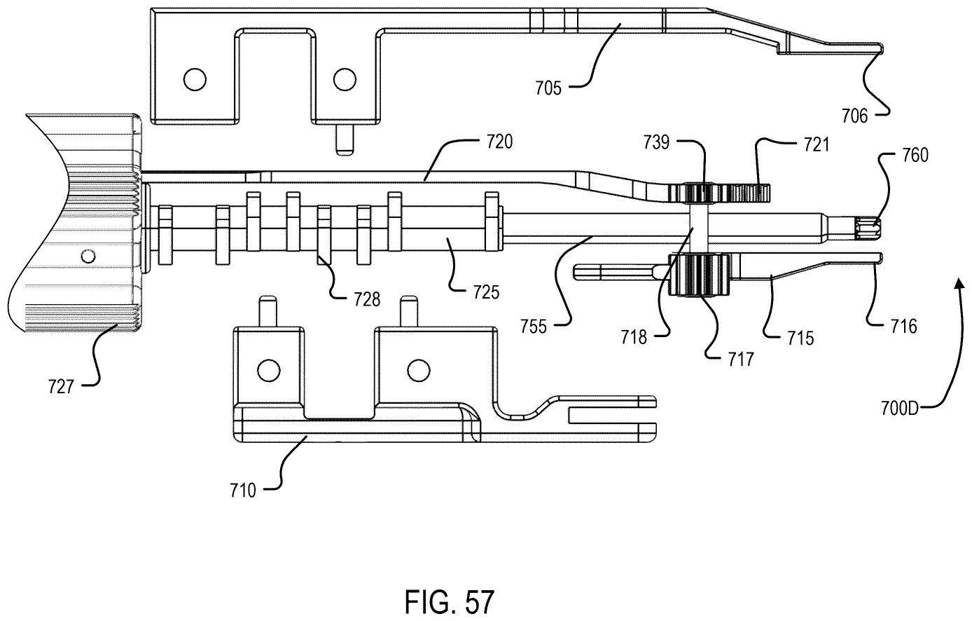

With reference to FIG. 55 and the exploded parts views of FIGS. 56-58, inserter portion 701 of surgical tool 700 may include: a distal end comprising a non-rotating jaw 705 having an end 706 for gripping an implant, and a rotating jaw 715 having an end 716 for gripping an implant that is coupled to a rotating jaw slide 710. In various embodiments, the non-rotating jaw 705 and rotating jaw 715, together as a pair, may be referred as “parallel jaws” which may grip and expand corresponding portions of an intervertebral spinal implant. Additionally, the non-rotating jaw end 706 and rotating jaw end 716 may each include female recesses having a size and shape generally corresponding to a size and shape of gripping protrusions 429, 419 of implant 400 (see FIG. 31). Rotating Jaw 715 may be operatively coupled with rotating jaw gear 717 which allows for the rotating jaw 715 to move in a vertical direction (represented by arrow in FIG. 55) along the inner gear thread of rotating jaw 715. Additionally, rotating jaw gear 717 is meshed with distal dial indicator gear 739, which is operatively coupled to distal gear rack teeth 721 on gear rack 720, such that when rotation knob handle 740 is rotated the rotating jaw gear 717 and distal dial indicator gear 739 are simultaneously activated. In this way, each incremental movement of rotation knob handle 740 causes a corresponding incremental movement of the dial indicator gear 739 such that throughout the entirety of an expansion process a surgeon knows exactly what angle of inclination the implant is in (e.g., lordosis). In various embodiments, distal dial indicator gear 739 may have a suitable diameter and number of teeth that are coordinated with respect to an implant having a particular size. For example, in the example embodiment, distal dial indicator gear 739 may be smaller than rotating jaw gear 717.

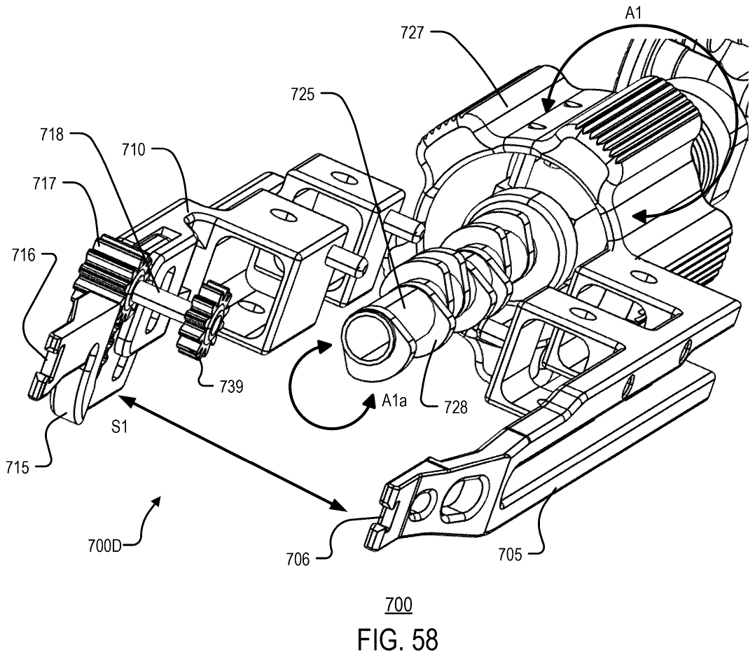

In the example embodiment shown in FIG. 58, twist grip 727 may be actionable to rotate clockwise and counterclockwise along the A1 rotation axis. Rotation thereof may effectuate the opening of non-rotating jaw 705 and rotating jaw 715 for gripping of the implant. For example, rotation of twist grip 727 may rotate the cam shaft 725 in the corresponding A1a direction such that the cam shaft lobes 728 laterally push rotating jaw 715 in a horizontal direction S1 away from non-rotating jaw 705. For example, by moving one of the jaws away from the other jaw, or even by moving both of the jaws away from one another. In various embodiments, the cam shaft lobes 728 may provide bilateral contact with non-rotating jaw 705 and rotating jaw slide 710 to precisely control the horizontal position. In some embodiments, the cam shaft lobes 728 are configured so that the “open” and “closes” jaw orientations are in a lock step with the orientation of the cam shaft 725. By moving the jaws apart from one another a horizontal distance between the two jaws is increased and an implant may be positioned between the two jaws for coupling to the jaws once the jaws are closed back together. In the “open jaw” orientation, gripping protrusions 419, 429 of implant 400 may be positioned horizontally in-line with the non-rotating jaw end 706 and rotating jaw end 716 such that the parallel jaws may be subsequently closed to the original position. For example, by rotating the cam twist grip 727 to retract the non-rotating jaw 705 and rotating jaw 715 to a “closed jaw” orientation thereby securely coupling surgical tool 700 with implant 400. In this way, the gripping protrusion 419 on superior endplate of implant 400 may be coupled with rotating jaw end 716 and the gripping protrusion 429 on inferior endplate of implant 400 may be coupled with non-rotating jaw 706 (see FIG. 31 showing implant). In the example embodiment, the dovetail recesses of jaw ends 706, 716 may mate with the dovetail gripping protrusions 419, 429 of implant. In various embodiments, the recess of rotating jaw end 716 may be larger than a recess of non-rotating jaw end 706. This may help ensure the implant is mated to the surgical tool in a proper orientation, e.g., it isn't mated in reverse. In some embodiments, rotating jaw end 716 may be coupled to gripping protrusion 419 on the superior endplate of implant 400 and non-rotating jaw end 706 may be coupled to gripping protrusion 429 on the inferior endplate of implant 400.

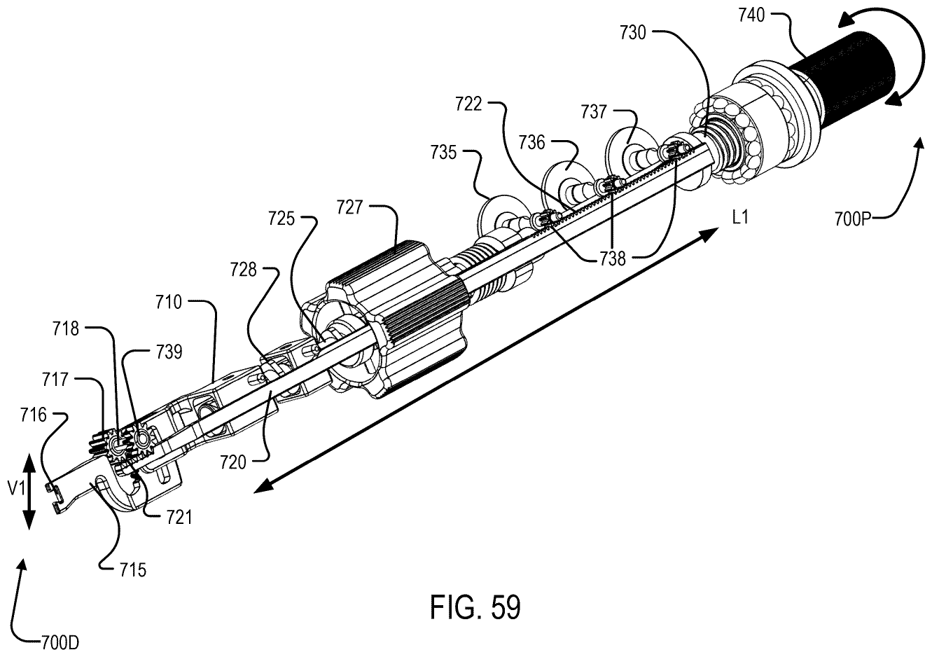

With reference to FIG. 59, once implant 400 is securely coupled with the surgical tool 700 via non-rotating jaw end 706 and rotating jaw end 716, the rotating jaw 715 may be translated vertically to expand the implant. In various embodiments, actuation of the rotation knob handle 740 may rotate the rotating jaw gear 717. As seen in FIG. 59, rotation knob handle 740 may be actionable to rotate along the A2 axis which translates gear rack 720 in a longitudinal direction L1 (see FIG. 59). In turn the translated gear rack 720 activates and rotates distal dial indicator gear 739 along distal gear rack teeth 721 which, in turn, activates the rotating jaw gear 717 that is coupled to distal dial indicator gear 739 by gear shaft 718 and translates the rotating jaw 715 in a vertical V1 direction. In this way, surgical tool 700 may cause pivoting of the superior endplate 410 of implant 400 along a hinge member 440 of the inferior endplate 420. For example, by lifting up on the superior endplate 410 via rotating jaw 715 the implant 400 may expand into an angled configuration because of the pivoting hinge type connection.

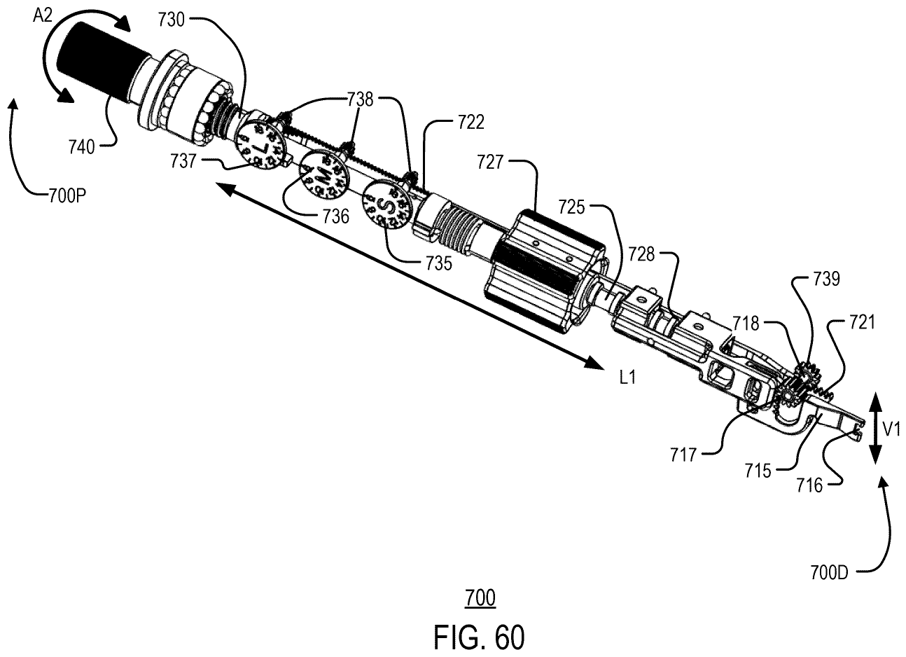

With reference to FIG. 59 and FIG. 60, when rotation knob handle 740 is rotated along rotation axis A2, the proximal gear rack teeth 722 and distal gear rack teeth 721 of the gear rack 720 are translated longitudinally in the L1 direction thereby simultaneously rotating each of the three proximal dial indicator gears 738 and the distal dial indicator gear 739. Additionally, in this embodiment distal dial indicator gear 739 is coupled to rotating jaw gear 717 via gear shaft 718 (see FIG. 59-60). In this way rotation of gears 717 and 739 translates the rotating jaw 715 vertically in the V1 direction thereby expanding the implant 400. In this embodiment, three proximal dial indicator gears 738 are individually attached to respective dial angle indicators for visually identifying and verifying an angle of inclination of a correspondingly sized implant 400. The respective dial indicators each identify the angle of inclination for a differently sized implant 400. In the example embodiment, three dial indicators are provided: a large dial angle indicator 737, a medium dial angle indicator 736, and a small dial angle indicator 735 although any number of indicators may be provided. Consistent with the disclosure herein, implants 400 of various sizes may be used depending on patient specific anatomy and the intended use case. The configuration of three different dial indicator gears 738 allows a surgeon to know the angle of inclination of any of the three differently sized implants. For example, the same relative movement of rotation knob handle 740 may cause different amounts of expansion and angulation for implants of different sizes, e.g., small, medium, large, and the disclosed surgical tool 700 can be used interchangeably with any of these differently sized implants.

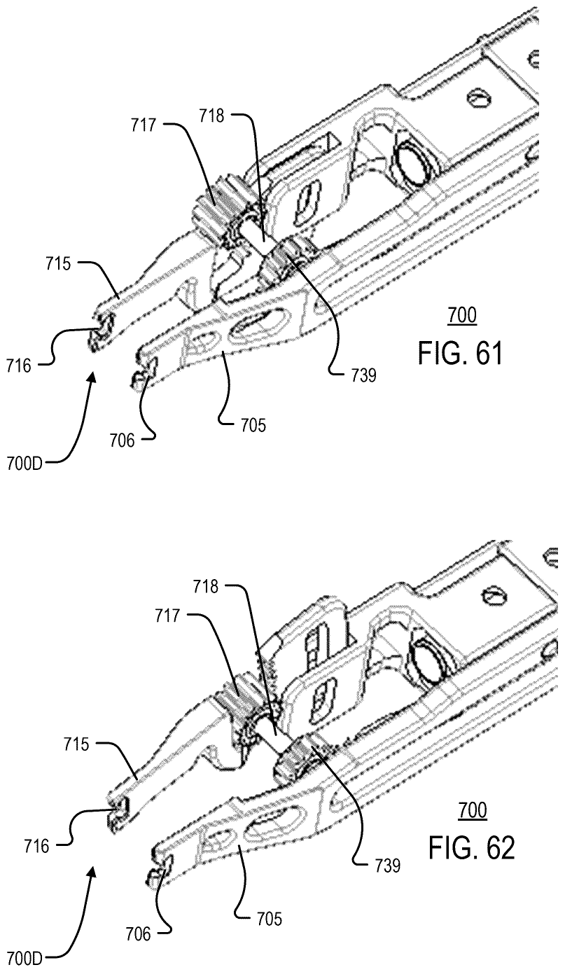

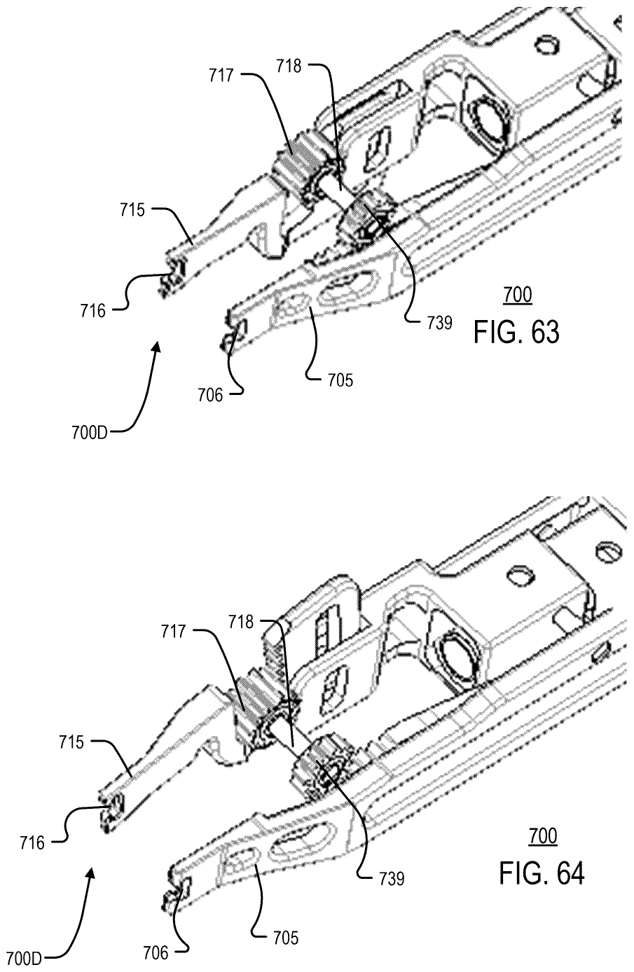

With reference to FIGS. 61-64, four different configurations for non-rotating jaw 705 and rotating jaw 715 are disclosed. FIG. 61 shows two parallel jaws in a closed position where the jaws are closer together and with rotating jaw 715 in a down position (neutral position). FIG. 62 shows the parallel jaws in a closed position where the jaws are closer together and with rotating jaw 715 in an up position for expanding the implant. FIG. 63 shows the parallel jaws in an open position where the jaws are farther apart for coupling to an implant and with rotating jaw 715 in a down position. FIG. 64 shows the parallel jaws in an open position where the jaws are farther apart for coupling to an implant and with rotating jaw 715 in an up position.

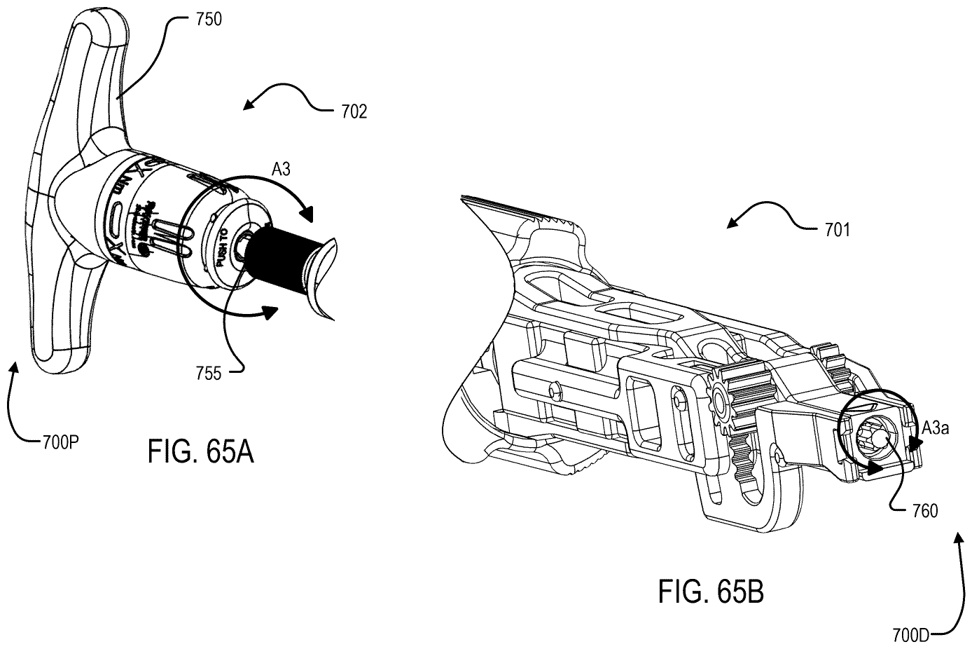

With reference to FIGS. 65A and 65B, the interaction between the drive portion and the inserter portion of surgical tool 700 will be discussed. As explained previously, drive portion 702 may be inserted within a central aperture extending in the longitudinal direction of inserter portion 701 such that the drive portion 702 may interact with the locking screw of implant 400Z. In the example illustration, proximal end of drive shaft 755 is securely coupled to drive actuator 750. Drive shaft 755 extends longitudinally through the apertures of rotation knob handle 740, proximal gear rack end 723, gear rack tube 730, and cam shaft 725 such that end 760 is disposed between the parallel jaws. This configuration allows a surgeon to expand implant 400Z and rotate the locking screw 50 of implant 400Z while it is expanded.

A method of operation for expanding an intervertebral implant to a desired angle of inclination will now be discussed. First, a surgeon may securely couple an intervertebral implant 400Z to surgical tool 700. Once implant 400Z is in the operatively engaged position with surgical tool 700, i.e., parallel jaws side by side in a closed position, a surgeon may insert implant 400Z into a disc space of a patient between a superior (cephalad) vertebra and inferior (caudal) vertebra, for example. After the implant 400Z is positioned between the superior and inferior vertebrae, the surgeon may rotate rotation knob handle 740 along the A2 axis thereby separating the superior endplate 410 apart from the inferior endplate 420, e.g., by translating the rotating jaw 715 vertically relative to the non-rotating jaw 705. In various embodiments, depending on the size of implant 400, the expanded angle may be identified by one of large dial angle indicator 737, medium dial angle indicator 736, or small dial angle indicator 735.

A method of locking an intervertebral implant in a desired angle of inclination will now be discussed. With the implant 400Z positioned and expanded appropriately in the disc space the surgeon may lock the superior endplate 410 and inferior endplate 420 in the desired configuration while surgical tool 700 maintains the intervertebral implant at a particular lordotic angle. At this stage, the surgeon may insert the drive portion 702 into the inserter portion 701 if they are not otherwise already together. It shall be understood that in some embodiments the drive portion 702 may remain in the inserter portion 701 throughout the insertion and expansion steps discussed above in the preceding paragraph. Next, the surgeon may verify that drive end 760 is matingly positioned in a corresponding drive end of the locking screw 50. Thereafter, the surgeon may activate the drive actuator 750 (rotate the T-shaped handle) to rotate drive shaft 755 along rotation axis A3 thereby rotating the drive end 760 and locking screw 50 because of the way drive feature 453B is engaged with drive end 760. For example, drive end 760 is rotated along axis A3a thereby causing rotation of the locking screw 50. In some embodiments, the non-rotating jaw 705 having an end 706 coupled to the gripping protrusion 429 on the inferior endplate of implant 400 may provide a two-part connection that resists rotation of the implant along a longitudinal axis of the drive shaft when drive end 760 provides rotational forces to actuate the rotation of the locking screw 50. The surgeon may continue to rotate the drive shaft 755 and the locking screw 50 until it advances sufficiently far enough into implant 400Z and locks the implant 400Z in an expanded configuration as explained previously. In some embodiments, drive portion 702 may include a torque limiting feature to ensure that the locking screw 50 is tight enough to hold the implant in the locked configuration but not too tight that shearing might occur. Once implant 400Z is inserted and opened to the desired configuration and locked, the drive shaft 755 may be disengaged from implant 400Z by removing drive actuator 750 and drive shaft 755 back through the aperture of rotation knob handle 740, proximal gear rack end 723, gear rack tube 730, and cam shaft 725 in a longitudinal direction from a distal end to a proximal end. Finally, the surgeon may rotate the cam twist grip 727 to horizontally open the non-rotating jaw 705 and rotating jaw 715 laterally away from one another in the S1 direction (see FIG. 58) to disengage from gripping protrusions 419, 429 of implant 400 and remove the inserter portion 701 of the surgical tool 700. Those with skill in the art will appreciate that the spreading of the parallel jaws laterally away from one another vs. vertically away from one another is critical to prevent the jaws from contacting delicate patient tissue or being obstructed by the adjacent superior and inferior vertebra during the uncoupling process.

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

What is claimed is:

1. An expandable implant movable between a contracted position and an expanded position within a disc space, comprising:
an expandable body extending from a proximal end to a distal end in a proximal-to-distal direction and extending from a first lateral side to a second lateral side in a widthwise direction, the expandable body being defined by a superior endplate and an inferior endplate that are hingedly connected;
the superior endplate comprises a channel located adjacent the distal end and extending in the widthwise direction from a first lateral side to a second lateral side of the superior endplate, and a first core having a screw slot and a distal engagement surface;
the inferior endplate comprises a rail located adjacent the distal end and extending in the widthwise direction from a first lateral side to a second lateral side of the inferior endplate, the rail having a size and shape generally corresponding to a size and shape of the channel and being located within the channel, and a second core having a proximal engagement surface; and
a locking screw extending in a longitudinal direction from a proximal end to a distal end, being extendable through the first core and the second core, and being movable between a locked position and an unlocked position,
wherein, in the locked position, the locking screw urges the distal engagement surface of the first core against the proximal engagement surface of the second core, and
wherein the superior endplate and inferior endplate are each a single unitary piece, respectively.

2. The expandable implant of claim 1, further comprising: a gripping portion located at the proximal end.

3. The expandable implant of claim 2, wherein the gripping portion comprises a first indentation and a second indentation for coupling to an inserter.

4. The expandable implant of claim 1, wherein:
the superior endplate comprises a first plurality of engagement features that are angled diagonally in a first direction from the first lateral side of the superior endplate by 20 degrees to 40 degrees with respect to a proximal surface of the superior endplate and a second plurality of engagement features that are angled diagonally in a second direction from the second lateral side of the superior endplate by 20 degrees to 40 degrees with respect to a proximal surface of the superior endplate, and
the inferior endplate comprises a first plurality of engagement features that are angled diagonally in a third direction from the first lateral side of the inferior endplate by 20 degrees to 40 degrees with respect to a proximal surface of the inferior endplate and a fourth plurality of engagement features that are angled diagonally in a fourth direction from the second lateral side of the inferior endplate by 20 degrees to 40 degrees with respect to a proximal surface of the superior endplate.

5. The expandable implant of claim 4, wherein:
the first plurality of engagement features and the second plurality of engagement features each comprise flattened top rails that are sequentially spaced apart with rounded bottom valleys therebetween, and
an imaginary extension of the flattened top rails extending in the first direction intersects with an imaginary extension of the flattened top rails extending in the second direction.

6. The expandable implant of claim 1, wherein:
the distal engagement surface of the first core comprises a first curved surface;
the proximal engagement surface of the second core comprises a second curved surface; and
the first curved surface is defined by a radius of a circle having a center point that is offset from an axis of rotation of the rail.

7. The expandable implant of claim 1, wherein:
the superior endplate comprises at least one hook portion adjacent the channel;
the inferior endplate comprises at least one relief portion adjacent the rail;
the at least one hook portion has a size and shape generally corresponding to a size and shape of the at least one relief portion; and
the at least one hook portion is disposed within the at least one relief portion.

8. The expandable implant of claim 1, wherein:
the locking screw comprises a threaded distal end and a drive feature;
the first core is disposed proximally with respect to the second core; and
the locking screw is threadably engaged with at least one of the first core and second core.

9. The expandable implant of claim 1, further comprising:
a surgical tool configured for moving the expandable implant from the contracted position to the expanded position and for rotating the locking screw between the locked position and the unlocked position, the surgical tool being configured to simultaneously rotate the locking screw into the locked position while supporting the expandable implant in the expanded position,
the surgical tool comprising:
an inserter portion configured to couple to and uncouple from the expandable implant, the inserter portion having: a rotation knob handle, a cam twist grip, and a first jaw and a second jaw configured to retain the expandable implant during expansion of the expandable implant in situ, and wherein a hollow shaft extends longitudinally through the inserter portion; and
a drive portion disposed within the hollow shaft of the inserter portion, the drive portion having a drive shaft extending from a proximal end to a distal end, the distal end including a drive portion configured to engage with the locking screw, and the proximal end including a drive actuator for rotating the drive shaft,
wherein actuation of the rotation knob handle pivotally expands the expandable implant, and
wherein actuation of the cam twist grip spreads the first jaw and the second jaw apart from one another in a transverse direction for coupling to the expandable implant.

10. A system, comprising:
an expandable implant movable between a contracted position and an expanded position, the expandable implant comprising:
an expandable body extending from a proximal end to a distal end in a proximal-to-distal direction and extending from a first lateral side to a second lateral side in a widthwise direction, the expandable body being defined by a superior endplate and an inferior endplate that are hingedly connected;
the superior endplate comprising a first core having a screw slot and a distal engagement surface, the superior endplate being formed as a unitary single piece;
the inferior endplate comprising a second core having a proximal engagement surface, the inferior endplate being formed as a unitary single piece; and
a locking screw movable between a locked position and an unlocked position;
wherein, in the locked position, the locking screw urges the distal engagement surface of the first core against the proximal engagement surface of the second core; and
a surgical tool configured to move the implant from the contracted position to the expanded position and to move the locking screw between the locked position and the unlocked position, the surgical tool being capable of moving the locking screw into the locked position while supporting the implant in the expanded position,
wherein the surgical tool further comprises:
a body portion having an aperture extending therethrough;
an outer shaft extending through the body portion and having a drive feature at a distal end thereof for rotating the locking screw from the unlocked position to the locked position; and
an inner shaft extending through the outer shaft and having a first thread pattern at an end thereof for drawing the implant towards the surgical tool.

11. The system of claim 10, wherein:
the expandable implant further comprises a first gripping indentation and a second gripping indentation disposed on the proximal end;
the surgical tool comprises a first gripping protrusion having a size and shape generally corresponding to a size and shape of the first gripping indentation; and
the surgical tool comprises a second gripping protrusion having a size and shape generally corresponding to a size and shape of the second gripping indentation.

12. The system of claim 11, wherein:
the first gripping indentation comprises a slot having a superior curved surface and an inferior curved surface; and
the second gripping indentation comprises a slot having a superior curved surface and an inferior curved surface.

13. The system of claim 10, wherein the surgical tool comprises a linkage assembly.

14. The system of claim 13, wherein the linkage assembly includes a first arm and a second arm.

15. The system of claim 14, wherein the surgical tool further comprises an actuator for causing the linkage assembly to pivotally expand the implant.

16. The system of claim 10, wherein:
the superior endplate further comprises a channel located adjacent the distal end and extending in the widthwise direction; and
the inferior endplate further comprises a rail located adjacent the distal end and extending in the widthwise direction, the rail having a size and shape generally corresponding to a size and shape of the channel and being located within the channel,
wherein the channel and rail each have an arcuate shape.

17. The system of claim 16, wherein:
the distal engagement surface of the first core comprises a first curved surface;
the proximal engagement surface of the second core comprises a second curved surface; and
the first curved surface is defined by a radius of a circle having a center point that is offset from an axis of rotation of the rail.

18. A system including an expandable implant movable between a contracted position and an expanded position and an inserter, comprising:
an expandable implant comprising:
an expandable body extending from a proximal end to a distal end in a proximal-to-distal direction and extending from a first lateral side to a second lateral side in a widthwise direction, the expandable body being defined by a superior endplate and an inferior endplate that are hingedly connected;
the superior endplate comprises a channel located adjacent the distal end and extending in the widthwise direction from a first lateral side to a second lateral side of the superior endplate, and a first core having a screw slot and a distal engagement surface;
the inferior endplate comprises a rail located adjacent the distal end and extending in the widthwise direction from a first lateral side to a second lateral side of the inferior endplate, the rail having a size and shape generally corresponding to a size and shape of the channel and being located within the channel, and a second core having a proximal engagement surface; and
a locking screw extending in a longitudinal direction from a proximal end to a distal end, being extendable through the first core and the second core, and being movable between a locked position and an unlocked position;
wherein, in the locked position, the locking screw urges the distal engagement surface of the first core against the proximal engagement surface of the second core; and
wherein the superior endplate and inferior endplate are each formed by a single unitary piece, respectively;
an inserter configured for inserting the expandable implant into a disc space of a patient and expanding the expandable implant in situ within the disc space, the inserter extending lengthwise in a longitudinal direction from a proximal end to a distal end, widthwise in a horizontal direction transverse to the longitudinal direction, and heightwise in a vertical direction transverse to the longitudinal direction and the horizontal direction, the inserter comprising:
a first jaw and a second jaw disposed at a distal end of the inserter and configured to couple to and uncouple from a first gripping protrusion and a second gripping protrusion of the expandable implant, respectively;
a first rotatable grip configured to expand the expandable implant; and
a second rotatable grip configured to spread the first jaw and the second jaw apart in the horizontal direction for coupling to the first gripping protrusion and second gripping protrusion;
wherein the first jaw comprises a first gripping indent having a size and shape corresponding to the first gripping protrusion and the second jaw comprises a second gripping indent having a size and shape corresponding to the second gripping protrusion.

19. The system of claim 18, further comprising:
a rack extending in the longitudinal direction and including teeth, the rack being operatively coupled to the first rotatable grip; and
a first gear meshed with the teeth of the rack and a second gear meshed with teeth of the first jaw,
wherein the rack is configured to move forward and backward in the longitudinal direction upon actuation of the first rotatable grip thereby rotating the first gear and second gear and moving the first jaw away from the second jaw in the vertical direction.

20. The system of claim 19, further comprising:
at least one indicator gear meshed with the teeth of the rack and being configured to rotate upon movement of the rack in the longitudinal direction; and
wherein the at least one indicator gear is coupled to a corresponding indicator configured to visually display an angle of inclination measured between the superior endplate and the inferior endplate of the expandable implant.
